SEQUENCE LISTING (25 sequences)

SEQ ID NO: 1 - protein, 454 aa, Artificial sequence
  Synthetic polypeptide
  misc_feature: amino acid sequence of FSYR
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLIPNLYNYLRKLDRALPDPIKIFEIGPCYRKESDGKEHLEEFTMLTFIQMGSGCTRENLESIITDFLNHLGIDFKIVGDSCMVLGDTLDVMHGDLELSSAVVGPIPLDREWGIDKPKIGAGFGLERLLKVKHDFKNIKRAARSESYYNGISTNL

SEQ ID NO: 2 - DNA, 1365 bp, Artificial sequence
  Synthetic polynucleotide
  misc_feature: nucleid acid sequence of FSYR
atggataaaaagcctttgaacactctgatttctgcgaccggtctgtggatgtcccgcaccggcaccatccacaaaatcaaacaccatgaagttagccgttccaaaatctacattgaaatggcttgcggcgatcacctggttgtcaacaactcccgttcttctcgtaccgctcgcgcactgcgccaccacaaatatcgcaaaacctgcaaacgttgccgtgttagcgatgaggacctgaacaaattcctgaccaaagctaacgaggatcagacctccgtaaaagtgaaggtagtaagcgctccgacccgtactaaaaaggctatgccaaaaagcgtggcccgtgccccgaaacctctggaaaacaccgaggcggctcaggctcaaccatccggttctaaattttctccggcgatcccagtgtccacccaagaatctgtttccgtaccagcaagcgtgtctaccagcattagcagcatttctaccggtgctaccgcttctgcgctggtaaaaggtaacactaacccgattactagcatgtctgcaccggtacaggcaagcgccccagctctgactaaatcccagacggaccgtctggaggtgctgctgaacccaaaggatgaaatctctctgaacagcggcaagcctttccgtgagctggaaagcgagctgctgtctcgtcgtaaaaaggatctgcaacagatctacgctgaggaacgcgagaactatctgggtaagctggagcgcgaaattactcgcttcttcgtggatcgcggtttcctggagatcaaatctccgattctgattccgctggaatacattgaacgtatgggcatcgataatgataccgaactgtctaaacagatcttccgtgtggataaaaacttctgtctgcgtccgatgctgattccgaacttgtacaactatttacgtaaactggaccgtgccctgccggacccgatcaaaatattcgagatcggtccttgctaccgtaaagagtccgacggtaaagagcacctggaagaattcaccatgctgacattcattcagatgggtagcggttgcacgcgtgaaaacctggaatccattatcaccgacttcctgaatcacctgggtatcgatttcaaaattgttggtgacagctgtatggtgttaggcgatacgctggatgttatgcacggcgatctggagctgtcttccgcagttgtgggcccaatcccgctggatcgtgagtggggtatcgacaaacctaaaatcggtgcgggttttggtctggagcgtctgctgaaagtaaaacacgacttcaagaacatcaaacgtgctgcacgttccgagtcctattacaatggtatttctactaacctgtaa

SEQ ID NO: 3 - protein, 454 aa, Artificial sequence
  Synthetic polypeptide
  misc_feature: wild type amino acid sequence of Methanosarcina mazei PylRS
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 4 - DNA, 69 bp, Artificial sequence
  Synthetic polynucleotide
  misc_feature: nucleic acid sequenc of tRNA_Pyl_CUA
ggaaacctgatcatgtagatcgaatggactctaaatccgttcagccgggttagattcccggggtttccg

SEQ ID NO: 5 - DNA, 36 bp, Artificial sequence
  Synthetic polynucleotide
ctaacaggaggaattagatctatggataaaaagcct

SEQ ID NO: 6 - DNA, 38 bp, Artificial sequence
  Synthetic polynucleotide
gatgatgatgatgatggtcgacttacaggttagtagaa

SEQ ID NO: 7 - DNA, 24 bp, Artificial sequence
  Synthetic polynucleotide
tatgccatggataaaaagcctttg

SEQ ID NO: 8 - DNA, 25 bp, Artificial sequence
  Synthetic polynucleotide
ctatgctagcttacaggttagtaga

SEQ ID NO: 9 - DNA, 24 bp, Artificial sequence
  Synthetic polynucleotide
aacgcggaactatcagtcgccggc

SEQ ID NO: 10 - DNA, 24 bp, Artificial sequence
  Synthetic polynucleotide
aacaaagaactatcagtcgccggc

SEQ ID NO: 11 - DNA, 24 bp, Artificial sequence
  Synthetic polynucleotide
aactgcgaactatcagtcgccggc

SEQ ID NO: 12 - DNA, 24 bp, Artificial sequence
  Synthetic polynucleotide
aacagcgaactatcagtcgccggc

SEQ ID NO: 13 - DNA, 24 bp, Artificial sequence
  Synthetic polynucleotide
aacaccgaactatcagtcgccggc

SEQ ID NO: 14 - DNA, 24 bp, Artificial sequence
  Synthetic polynucleotide
aaccatgaactatcagtcgccggc

SEQ ID NO: 15 - DNA, 29 bp, Artificial sequence
  Synthetic polynucleotide
gaacgcgttgtctaccatggtatatctcc

SEQ ID NO: 16 - DNA, 41 bp, Artificial sequence
  Synthetic polynucleotide
ccatggtagacaacgcgttcaactatgaactatcagtcgcc

SEQ ID NO: 17 - DNA, 43 bp, Artificial sequence
  Synthetic polynucleotide
tatatctccttcttaaagttaaacaaaattatttctagagggg

SEQ ID NO: 18 - DNA, 27 bp, Artificial sequence
  Synthetic polynucleotide
aactatgactagtcatgaccaactgac

SEQ ID NO: 19 - DNA, 36 bp, Artificial sequence
  Synthetic polynucleotide
cgcatacgcgtccgcctacgctctagccatcatagt

SEQ ID NO: 20 - DNA, 43 bp, Artificial sequence
  Synthetic polynucleotide
tggctagagcgtaggcggacgcgtatgcggaagaggaaatccg

SEQ ID NO: 21 - DNA, 29 bp, Artificial sequence
  Synthetic polynucleotide
ccaagctcagcttattagtgatggtgatg

SEQ ID NO: 22 - DNA, 29 bp, Artificial sequence
  Synthetic polynucleotide
tatacatatgtccaaactcgatctaaacg

SEQ ID NO: 23 - DNA, 53 bp, Artificial sequence
  Synthetic polynucleotide
agccaagcttttaatgatgatgatgatgatgcccttcgtgtaacccacattcc

SEQ ID NO: 24 - DNA, 24 bp, Artificial sequence
  Synthetic polynucleotide
gaacatcgattagaaccctggcac

SEQ ID NO: 25 - DNA, 21 bp, Artificial sequence
  Synthetic polynucleotide
agttttgcaacggtcagtttg

(19) United States
(12) Patent Application Publication
(10) Pub. No.: US 2021/0002325 A1
(43) Pub. Date: Jan. 7, 2021

(54) BIOREACTIVE COMPOSITIONS AND METHODS OF USE THEREOF

(71) Applicant: The Regents of the University of California, Oakland, CA (US)

(72) Inventors: Lei Wang, San Francisco, CA (US); Nanxi Wang, San Francisco, CA (US)

(21) Appl. No.: 16/977,439

(22) PCT Filed: Mar. 8, 2019
(86) PCT No.: PCT/US2019/021433

§ 371 Date: Sep. 1, 2020

Related U.S. Application Data
(60) Provisional application No. 62/640,450, filed on Mar. 8, 2018.

(51) Int. Cl. C07K 1/107; C12N 9/00; C12P 21/02

(52) U.S. Cl. CPC C07K 1/1072; C12P 21/02; C12N 9/93

(57) ABSTRACT

Provided herein are, inter alia, non-toxic, bioreactive unnatural amino acids and methods of using same.

### CROSS-REFERENCE TO RELATED APPLICATIONS

This application claims priority to U.S. Application No. 62/640,450 filed Mar. 8, 2018, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with government support under grant nos. R01 GM118384 and MH114079 awarded by the National Institutes of Health. The government has certain rights in the invention.

### BACKGROUND

Amino acid side chains of proteins usually cannot form covalent bonds with each other except cysteine, which generates the weak and reversible disulfide bond. Therefore, proteins use primarily noncovalent interactions within or between proteins. A latent bioreactive unnatural amino acid that is nontoxic to cells and able to react with multiple natural amino acid residues would dramatically expand the diversity of proteins amenable to covalent bonding in vivo. By expanding the diversity of proteins amenable to covalent bonding in vivo it is possible to enhance existing protein properties or evolve new functions through harnessing the novel covalent linkages. In addition, the ability to form covalent linkages between proteins would allow irreversible capture of protein-protein interactions in vivo, which can be useful for protein identification, drug target discovery, or biotherapeutics.

Provided herein are, inter alia, solutions to these and other needs in the art.

### SUMMARY

In an aspect is provided a biomolecule conjugate including a first biomolecule moiety conjugated to a second biomolecule moiety through a bioconjugate linker, wherein the bioconjugate linker has the formula:

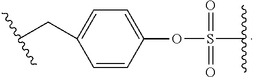 [SMILES: *Cc1ccc(OS(*)(=O)=O)cc1]

In an aspect is provided a protein having the unnatural amino acid side chain of:

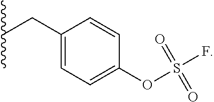 [SMILES: *Cc1ccc(OS(=O)(=O)F)cc1]

In aspects, the protein further comprises a lysine, histidine, tyrosine, or a combination of two or more thereof that is proximal to this unnatural amino acid side chain.

In an aspect is provided a protein of Formula (I):

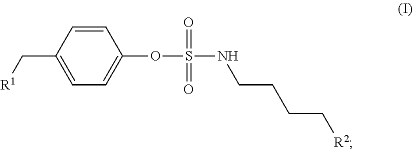

wherein R¹ and R² are each independently a peptidyl moiety.

In an aspect is provided a protein of Formula (II):

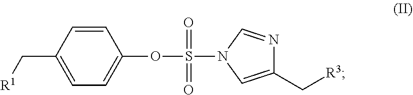

wherein R¹ and R² are each independently a peptidyl moiety.

In an aspect is provided a protein of Formula (III):

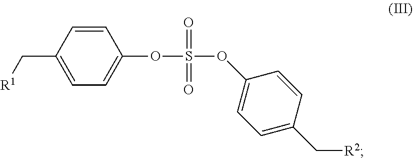

wherein R¹ and R² are each independently a peptidyl moiety.

In an aspect is provided a protein comprising a moiety of Formula (A), a moiety of Formula (B), a moiety of Formula (C), or a combination of two or more thereof:

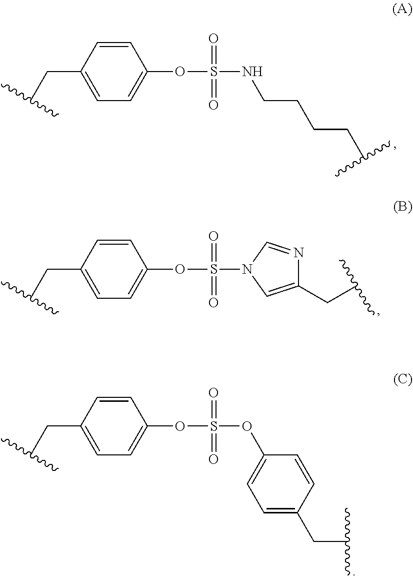 [SMILES: *CCCCNS(=O)(=O)Oc1ccc(C*)cc1 ; *Cc1ccc(OS(=O)(=O)n2cnc(C*)c2)cc1 ; *Cc1ccc(OS(=O)(=O)Oc2ccc(C*)cc2)cc1]

In an aspect is provided a pyrrolysyl-tRNA synthetase, including at least 5 amino acid residues substitutions within the substrate-binding site of the mutant pyrrolysyl-tRNA synthetase of SEQ ID NO:3.

In an aspect is provided a vector including a nucleic acid sequence encoding a mutant pyrrolysyl-tRNA synthetase as described herein.

In an aspect is provided a complex including a pyrrolysyl-tRNA synthetase as described herein, and fluorosulfate-L-tyrosine (FSY).

In an aspect is provided a cell that comprises fluorosulfate-L-tyrosine (FSY); a biomolecule conjugate as described herein; an FSY biomolecule as described herein; a pyrrolysyl-tRNA synthetase as described herein; a vector as described herein; or a complex as described herein. In aspects, the cell is a bacterial cell or a mammalian cell.

These and other embodiments and aspects of the disclosure are described in more detail herein.

### BRIEF DESCRIPTION OF THE DRAWINGS

FIGS. 1A-1E. Genetically encode FSY into proteins in E. coli. FIG. 1A: Structure of FSY. FIG. 1B: Scheme showing proximity-enabled SuFEx reaction between FSY and a natural nucleophilic residue (abbreviated as Nu). FIG. 1C: SDS-PAGE showing FSY incorporation into Afb(36TAG) in E. coli. FIG. 1D: ESI-TOF MS spectrum of intact Afb-36FSY. FIG. 1E: Tandem MS spectrum of Z-24FSY.

FIGS. 2A-2C. Genetically encode FSY into proteins in mammalian cells. FIG. 2A: FACS analysis of FSY incorporation into EGFP-182TAG in HeLa cells. FIG. 2B: Total EGFP fluorescence intensity measured from the same number of HeLa-EGFP-182TAG reporter cells. Error bar: s.e.m., n=6. FIG. 2C: Fluorescence images of HeLa-EGFP-182TAG reporter cells.

FIGS. 3A-3D. FSY crosslinks proximal Lys, His and Tyr via SuFEx directly in E. coli cells. FIG. 3A: Structure of Afb-Z complex showing two proximal sites for FSY and target residue X incorporation. FIG. 3B: Top: Western blot of E. coli cell lysates; Bottom: SDS-PAGE of proteins His-tag purified from E. coli. FIGS. 3C-3D: Tandem MS spectrum of MBP-Z-24FSY/Afb-7Lys (FIG. 3C) and MBP-Z-24FSY/Afb-7His (FIG. 3D).

FIGS. 4A-4C. FSY crosslinks Tyr via SuFEx intramolecularly in E. coli cells. FIG. 4A: Structure of CaM showing sites for FSY and target Tyr. (FIG. 4B) SDS-PAGE and (FIG. 4C) tandem MS spectrum of purified CaM-76FSY-80Tyr.

FIGS. 5A-5C. FSY crosslinks Tyr via SuFEx intermolecularly. (FIG. 5A) Structure of Trx1 in complex with PAPS reductase showing FSY site and the native Tyr191. (FIG. 5B) SDS-PAGE and (FIG. 5C) tandem MS spectrum of Trx1 crosslinked with PAPS reductase.

FIG. 6 provides a growth curve of E. coli DH10B cells at 37° C. in the presence or absence of 1 mM FSY. The experiments were repeated for three times.

FIG. 7 shows a FACS analysis of AzF incorporation into EGFP-182TAG HeLa reporter cells.

FIG. 8 shows a cell viability assay for HeLa-EGFP-182TAG reporter cells and 293T cells incubated with various concentrations of FSY. Error bars represent s.e.m.; n=3.

FIG. 9 shows an SDS-PAGE analysis of Trx62FSY crosslinking with PAPS reductase at pH 7.4 and 8.0.

FIG. 10 provides an illustration of FSY behavior in living cells.

FIG. 11 provides a ligand-receptor interface showing the site for FSY incorporation (Q68) on hGH and the target residue Lys166 on the hGH receptor

FIG. 12 is a Western blot analysis of hGH(FSY) binding with the extracellular domain of hGH receptor.

FIG. 13 is a Western blot analysis of pSTAT5 production in BAF3 cells upon stimulation by hGH(FSY) or hGH(WT), as described in Example 2.

### DETAILED DESCRIPTION

A latent bioreactive unnatural amino acid that is nontoxic to cells and able to react with multiple natural amino acid residues would dramatically expand the diversity of proteins amenable to covalent bonding in vivo. Described herein is a new tRNA/aminoacyl-tRNA synthetase pair to genetically encode fluorosulfate-L-tyrosine (FSY) into biomolecules (e.g., proteins) in live cells. FSY, which was found to be nontoxic to cells, can react with proximal lysine, histidine, and tyrosine in proteins both in vitro and in live cells.

Amino acid side chains of proteins usually cannot form covalent bonds with each other except cysteine, which generates the weak and reversible disulfide bond. Therefore, proteins primarily use noncovalent interactions within or between proteins. To endow proteins with covalent bonding ability, the inventors genetically incorporated the latent bioreactive unnatural amino acid fluorosulfate-L-tyrosine, which can selectively react with lysine, histidine, or tyrosine, forming covalent linkages within proteins and between proteins directly in vivo.

The genetically encoded fluorosulfate-L-tyrosine provides proteins with the ability to covalently bond by targeting multiple residues. When used within proteins, this is a novel protein engineering method to enhance existing protein properties or evolve new functions through harnessing the novel covalent linkages. When used between proteins, it can capture interacting proteins irreversibly, which can be useful for protein identification, drug target discovery, or biotherapeutics.

Existing technology, such as protein modification or bioorthogonal chemistry, can equip proteins with covalent bonding ability in vitro. The technology described herein, however, can arm proteins with covalent bonding ability in vivo. The final product can be developed in vivo (in live cells), which is advantageous for target identification with physiological relevance and for scale up production through recombinant approach.

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In aspects, monocyclic ring systems are cyclic hydrocarbon groups containing from 3 to 8 carbon atoms, where such groups can be saturated or unsaturated, but not aromatic. In aspects, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In aspects, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In aspects, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In aspects, the bridged or fused bicyclic cycloalkyl is attached to the parent molecular moiety through any carbon atom contained within the monocyclic cycloalkyl ring. In aspects, cycloalkyl groups are optionally substituted with one or two groups which are independently oxo or thia. In aspects, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In aspects, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In aspects, the multicyclic cycloalkyl is attached to the parent molecular moiety through any carbon atom contained within the base ring. In aspects, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In aspects, a cycloalkenyl is a monocyclic, bicyclic, or a multicyclic cycloalkenyl ring system. In aspects, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In aspects, bicyclic cycloalkenyl rings are bridged monocyclic rings or a fused bicyclic rings. In aspects, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In aspects, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In aspects, the bridged or fused bicyclic cycloalkenyl is attached to the parent molecular moiety through any carbon atom contained within the monocyclic cycloalkenyl ring. In aspects, cycloalkenyl groups are optionally substituted with one or two groups which are independently oxo or thia. In aspects, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In aspects, the multicyclic cycloalkenyl is attached to the parent molecular moiety through any carbon atom contained within the base ring. In aspects, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In aspects, heterocyclyl groups are optionally substituted with one or two groups which are independently oxo or thia. In certain aspects, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In aspects, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” or “—” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In aspects, the alkylarylene group has the formula:

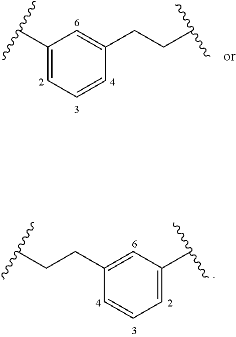 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In aspects, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (B) alkyl, heteroalkyl, cycloalkyl, heterocycloalkyl, aryl, heteroaryl, substituted with at least one substituent selected from: (i) oxo, halogen, —CCl₃, —CBr₃, —CF₃, —C₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl, heteroalkyl, cycloalkyl, heterocycloalkyl, aryl, heteroaryl, substituted with at least one substituent selected from: (a) oxo, halogen, —CCl₃, —CBr₃, —CF₃, —C₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in aspects, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In aspects, at least one or all of these groups are substituted with at least one size-limited substituent group. In aspects, at least one or all of these groups are substituted with at least one lower substituent group.

In embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In aspects of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In aspects, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In aspects, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In aspects, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In aspects, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In aspects, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In aspects, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In aspects, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. y way of example, a nonspecific nucleic acid sequence is a sequence of nucleic acid residues that does not function as an inhibitory nucleic acid when contacted with a cell or organism.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the UPAC-UB Biochemical Nomenclature Commission. Nucleotides, likewise, may be referred to by their commonly accepted single-letter codes.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In aspects, the amino acid side chain may be a non-natural amino acid side chain. In aspects, the amino acid side chain is H,

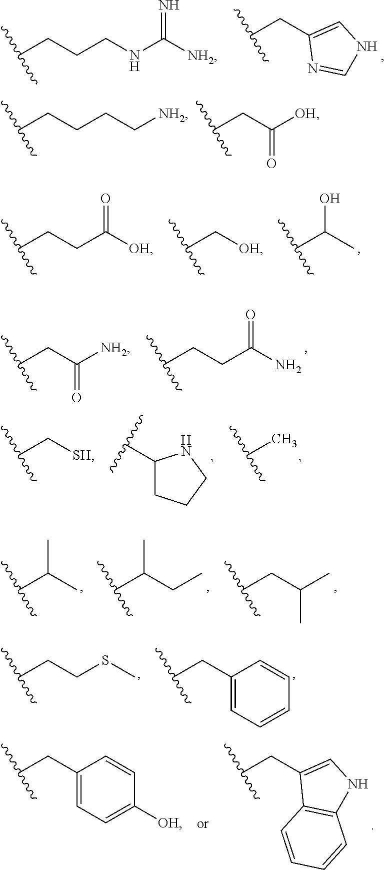

In embodiments, the unnatural amino acid side chain is

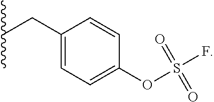 [SMILES: *Cc1ccc(OS(=O)(=O)F)cc1]

The term “non-natural amino acid side chain” or “unnatural amino acid side chain” or “Uaa” refers to the functional substituent of compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptanecarboxylic acid hydrochloride, cis-6-Amino-3-cyclohexene-1-carboxylic acid hydrochloride, cis-2-amino-2-methylcyclohexanecarboxylic acid hydrochloride, cis-2-amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine. In embodiments, the unnatural amino acid is fluorosulfate-L-tyrosine (FSY) having the following formula:

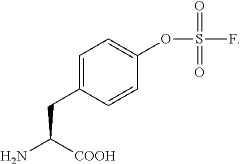 [SMILES: N[C@@H](Cc1ccc(OS(=O)(=O)F)cc1)C(=O)O]

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M). (see, e.g., Creighton, Proteins (1984)).

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to Ala302 of the PylRS protein when the selected residue occupies the same essential spatial or other structural relationship as Ala302 in the PylRS protein. In embodiments, where a selected protein is aligned for maximum homology with the PylRS protein, the position in the aligned selected protein aligning with Ala302 is said to correspond to Ala302. Instead of a primary sequence alignment, a three dimensional structural alignment can also be used, e.g., where the structure of the selected protein is aligned for maximum correspondence with the PylRS protein and the overall structures compared. In this case, an amino acid that occupies the same essential position as Ala302 in the structural model is said to correspond to the Ala302 residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “biomolecule” as used herein refers to large macromolecules such as, for example, proteins, carbohydrates, lipids, and nucleic acids, as well as small molecules such as, for example, primary and secondary metabolites. In aspects, the term biomolecule refers to a protein. In aspects, the term biomolecule refers to a nucleic acid. In aspects, the term biomolecule refers to a carbohydrate.

The term “biomolecule moiety” refers to a peptidyl moiety, a carbohydrate moiety, a lipid moiety, or a nucleic acid moiety that forms a biomolecule.

The term “peptidyl moiety” as used herein refers to a protein, protein fragment, or peptide that may form part of a biomolecule or a biomolecule conjugate. In aspects, the peptidyl moiety forms part of a biomolecule (e.g., protein). In aspects, the peptidyl moiety forms part of a biomolecule (e.g., protein) conjugate. The peptidyl moiety may also be substituted with additional chemical moieties (e.g., additional R substituents).

The term “carbohydrate moiety” as used herein refers to carbohydrates, for example, polyhydroxy aldehydes, ketones, alcohols, acids, their simple derivatives and their polymers having linkages of the acetal type, that may form part of a biomolecule or a biomolecule conjugate. In aspects, the carbohydrate moiety forms part of a biomolecule. In aspects, the carbohydrate moiety forms part of a biomolecule conjugate. The carbohydrate moiety may also be substituted with additional chemical moieties (e.g., additional R substituents).

The term “nucleic acid moiety” as used herein refers to nucleic acids, for example, DNA, and RNA, that may form part of a biomolecule or biomolecule conjugate. In aspects, the nucleic acid moiety forms part of a biomolecule. In aspects, the nucleic acid moiety forms part of a biomolecule conjugate. The nucleic acid moiety may also be substituted with additional chemical moieties (e.g., additional R substituents).

The term “pyrrolysyl-tRNA synthetase” refers to an enzyme (including homologs, isoforms, and functional fragments thereof) with pyrrolysyl-tRNA synthetase activity. Pyrrolysyl-tRNA synthetase is an aminoacyl-tRNA synthetase that catalyzes the reaction necessary to attach α-amino acid pyrrolysine to the cognate tRNA (tRNA^{pyl}), thereby allowing incorporation of pyrrolysine during proteinogenesis at amber stop codons (i.e., UAG). The term includes any recombinant or naturally-occurring form of pyrrolysyl-tRNA synthetase or variants, homologs, or isoforms thereof that maintain pyrrolysyl-tRNA synthetase activity (e.g. within at least 30%, 40%, 50%, 60%, 70%, 80%, 90%, 95%, or 100% activity compared to wild-type pyrrolysyl-tRNA synthetase). In aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring pyrrolysyl-tRNA synthetase. In aspects, the pyrrolysyl-tRNA synthetase comprises the sequence set forth by SEQ ID NO:3. In aspects, the pyrrolysyl-tRNA synthetase is the sequence set forth by SEQ ID NO:3.

The term “mutant pyrrolysyl-tRNA synthetase” or “mutant PylRS” refers to any pyrrolysyl-tRNA synthetase that has a different amino acid sequence from wild-type amino acid sequence of Methanosarcina mazeit pyrrolysyl-tRNA synthetase set forth as SEQ ID NO:3. In aspects, “mutant pyrrolysyl-tRNA synthetase” refers to any pyrrolysyl-tRNA synthetase that catalyzes the attachment of fluorosulfate-L-tyrosine (FSY) to a tRNA^{pyl}. In aspects, the mutant pyrrolysyl-tRNA synthetase includes the sequence set forth by SEQ ID NO:1. In aspects, the mutant pyrrolysyl-tRNA synthetase is the sequence set forth by SEQ ID NO:1. In aspects, the mutant pyrrolysyl-tRNA synthetase is encoded by the sequence set forth by SEQ ID NO:2. In aspects, “mutant pyrrolysyl-tRNA synthetase” is referred to as “pyrrolysyl-tRNA synthetase,” and the skilled artisan will readily recognize whether the pyrrolysyl-tRNA synthetase is mutant based on a comparison to the wild-type SEQ ID NO:3.

The term “tRNA^{Pyl}” and “tRNA_{CUA}^{Pyl}” (i.e., tRNA(superscript Pyl)(subscript CUA)) both refer to a single-stranded RNA molecule containing about 50 to about 100 nucleotides which fold via intrastrand base pairing to form a characteristic cloverleaf structure that carries a specific amino acid (e.g., pyrrolysine, FSY) and matches it to its corresponding codon (i.e., a complementary to the anticodon of the tRNA) on an mRNA during protein synthesis. In tRNA^{Pyl}, the anticodon is CUA. Anticodon CUA is complementary to amber stop codon UAG. The abbreviation “Pyl” of tRNA^{Pyl} stands for pyrrolysine and the “CUA” of tRNA^{Pyl} refers to its anticodon CUA. In aspects, tRNA^{Pyl} is attached to FSY. In aspects, tRNA^{Pyl} refers to a single-stranded RNA molecule containing about 70 to about 90 nucleotides.

The term “substrate-binding site” as used herein refers to residues located in the enzyme active site that form temporary bonds or interactions with the substrate. In aspects, the substrate-binding site of pyrrolysyl-tRNA synthetase refers to residues located in the active site of pyrrolysyl-tRNA synthetase that form temporary bonds or interactions with the amino acid substrate. In aspects, the substrate-binding site of pyrrolysyl-tRNA synthetase includes one or more of the following residues: alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO: 3.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The term “complex” refers to a composition that includes two or more components, where the components bind together to make a functional unit. In aspects, a complex described herein include a mutant pyrrolysyl-tRNA synthetase described herein and an amino acid substrate (e.g., FSY). In aspects, a complex described herein includes a mutant pyrrolysyl-tRNA synthetase described herein and a tRNA (e.g., tRNA^{Pyl}). In aspects, a complex described herein includes a mutant pyrrolysyl-tRNA synthetase described herein, an amino acid substrate (e.g., FSY) and a tRNA (e.g., tRNA^{Pyl}). In aspects, a complex described herein includes at least two components selected from the group consisting of a mutant pyrrolysyl-tRNA synthetase described herein, an amino acid substrate (e.g., FSY), a polypeptide containing FSY, and a tRNA (e.g., tRNA^{Pyl})

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In aspects, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Contacting” is used in accordance with its plain ordinary meaning and refers to the process of allowing at least two distinct species (e.g. chemical compounds including biomolecules, biomolecule moieties, or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be biomolecules and/or biomolecule moieties as described herein. In aspects, contacting includes allowing two biomolecule moieties as described herein to interact, wherein the biomolecule moieties covalently bond to form a conjugate.

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In aspects, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982. In aspects, the first bioconjugate reactive group (e.g., maleimide moiety) is covalently attached to the second bioconjugate reactive group (e.g. a sulfhydryl). In aspects, the first bioconjugate reactive group (e.g., haloacetyl moiety) is covalently attached to the second bioconjugate reactive group (e.g. a sulfhydryl). In aspects, the first bioconjugate reactive group (e.g., pyridyl moiety) is covalently attached to the second bioconjugate reactive group (e.g. a sulfhydryl). In aspects, the first bioconjugate reactive group (e.g., —N-hydroxysuccinimide moiety) is covalently attached to the second bioconjugate reactive group (e.g. an amine). In aspects, the first bioconjugate reactive group (e.g., maleimide moiety) is covalently attached to the second bioconjugate reactive group (e.g. a sulfhydryl). In aspects, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g. phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In aspects, the bioconjugate comprises a molecular entity derived from the reaction of an unsaturated bond, such as a maleimide, and a sulfhydryl group.

The terms “fluorosulfate-L-tyrosine” and “FSY” refer to the unnatural amino acid having the structure:

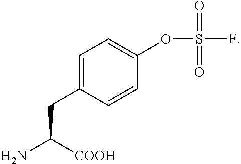 [SMILES: N[C@@H](Cc1ccc(OS(=O)(=O)F)cc1)C(=O)O]

FSY comprises the amino acid side chain of the formula:

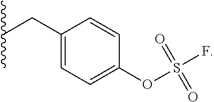 [SMILES: *Cc1ccc(OS(=O)(=O)F)cc1]

The term “FSY biomolecule” refers to a biomolecule comprising the FSY unnatural amino acid and/or the amino acid side chain thereof.

The term “biomolecule conjugate” refers to any biomolecule comprising a bioconjugate linker of the formula:

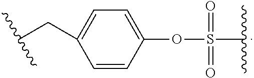 [SMILES: *Cc1ccc(OS(*)(=O)=O)cc1]

The term “FSY protein” refers to a protein comprising the FSY unnatural amino acid and/or the amino acid side chain thereof.

The term “protein conjugate” refers to any protein comprising a bioconjugate linker of the formula:

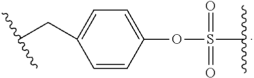 [SMILES: *Cc1ccc(OS(*)(=O)=O)cc1]

The term “sulfur-fluoride exchange reaction” or “SuFEx” refers to a type of click chemistry as described in detail by, e.g., Dong et al, Angewandte Chemie, 53(36):9340-9448 (2014); Wang et al, J. Am. Chem. Soc., 140(15):4995-4999 (2018); and as described in the examples herein. The term “proximally-enabled” SuFEx refers to the sulfur-fluoride exchange reaction occurring when the reactive species are proximal to each other, i.e., spatially close enough for the SuFEx reaction to occur. The proximity may occur within a single biomolecule (e.g., protein) or between two different biomolecules (e.g., proteins). The skilled artisan could readily determine whether the reactive species are sufficiently proximal for the reaction to occur (e.g., sulfur-fluoride exchange reaction between FSY and lysine, histidine, or tyrosine to form the bioconjugate, the moiety of Formula (A), (B), or (C), or the protein of Formula (I), (II), or (III)).

The term “intermolecular linker” refers to a linking group between two biomolecules. For example, when the moiety of Formula (A), (B), or (C) is an intermolecular linker, then the peptidyl moiety of R¹ is a first protein and the peptidyl moiety of R² is a second protein, such that the first protein and the second protein are covalently bonded via the moiety of Formula (A), (B), or (C). In aspects, the first protein and the second protein can be the same protein, e.g., providing an intermolecular linker between two proteins having the same amino acid sequence. In aspects, the first protein and the second protein can be different proteins, e.g., providing an intermolecular linker between two different proteins, such as a hormone and the receptor for the hormone.

The term “intramolecular linker” refers to a linking group within a biomolecule. For example, when the moiety of Formula (A), (B), or (C) is an intramolecular linker, then the peptidyl moiety of R¹ and the peptidyl moiety of R² are in the same protein. A compound having an intramolecular linker may also be referred to as an intramolecularly conjugated biomolecule conjugate or an intramolecularly conjugated biomolecule protein.

Biomolecules and Biomolecule Conjugates

Provided herein are biomolecules and biomolecule conjugates formed through the interaction of latent bioreactive unnatural amino acids with naturally occurring amino acids. fluorosulfate-L-tyrosine (FSY), a latent bioreactive unnatural amino acid, facilitates formation of covalent bonds with proximal target amino acid residues (e.g., lysine, histidine, tyrosine) by undergoing a click chemistry reaction (e.g., sulfur-fluoride exchange reaction (SuFEx)). For example, FSY may be inserted into or replace an amino acid in a naturally occurring protein, thereby endowing the protein with the ability to form a covalent bond with proximally positioned target amino acid residues (e.g., lysine, histidine, tyrosine) on the protein itself or with proteins it naturally interacts with. FSY may be used to facilitate the formation of covalent bonds between or within proteins in both in vitro and in vivo conditions, owing, at least in part, to its being non-toxic to cells. As such, the latent bioreactive unnatural amino acid FSY is useful for covalently linking biomolecules (e.g., proteins, carbohydrates, nucleic acids) to form biomolecule conjugates. In aspects, the latent bioreactive unnatural amino acid FSY is useful for covalently linking biomolecule moieties (e.g., peptidyl moieties) within a single biomolecule (e.g., protein). In aspects, the latent bioreactive unnatural amino acid FSY is useful for covalently linking biomolecule moieties (e.g., peptidyl moieties) in different biomolecules (e.g., covalently linking two proteins).

As shown herein, FSY, as a latent bioreactive unnatural amino acid, has shown excellent chemical functionality (i.e., superior properties) compared to previously described bioreactive unnatural amino acids. For example, FSY is stable, nontoxic and nonreactive inside cells, yet when placed in proximity to target residues it becomes reactive under cellular conditions. FSY is able to react with lysine, histidine, and tyrosine specifically with great selectivity via proximity-enabled SuFEx reaction within and between proteins under physiological conditions. No bioreactive unnatural amino acid has been reported that is nontoxic inside cells and is able to react with more than 2 amino acid residues.

Provided herein are biomolecules comprising one or more latent bioreactive unnatural amino acids. In aspects, the biomolecule is a protein, a nucleic acid, or a carbohydrate. In aspects, the biomolecule is a protein. In aspects, the latent bioreactive unnatural amino acid is fluorosulfate-L-tyrosine (FSY) having the formula:

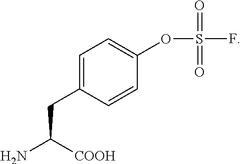 [SMILES: N[C@@H](Cc1ccc(OS(=O)(=O)F)cc1)C(=O)O]

In aspects, the biomolecule is a protein comprising the FYS unnatural amino acid. In aspects, the biomolecule is a protein comprising the FYS amino acid side chain represented by the formula:

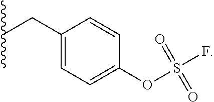 [SMILES: *Cc1ccc(OS(=O)(=O)F)cc1]

In aspects, the protein comprises FSY that is proximal to lysine, histidine, tyrosine, or a combination of two or more thereof. In aspects, the protein comprises FSY that is proximal to lysine. In aspects, the protein comprises FSY that is proximal to histidine. In aspects, the protein comprises FSY that is proximal to tyrosine. In aspects “proximal” means that FSY and lysine, histidine, or tyrosine are close enough to each other for a SuFEx reaction to successfully occur. In aspects, “proximal” means that FSY is within 1 to 20 amino acids of a lysine, histidine, or tyrosine. In aspects “proximal” means that FSY is within 1 to 15 amino acids of a lysine, histidine, or tyrosine. In aspects “proximal” means that FSY is within 1 to 10 amino acids of a lysine, histidine, or tyrosine. In aspects “proximal” means that FSY is within 1 to 9 amino acids of a lysine, histidine, or tyrosine. In aspects “proximal” means that FSY is within 1 to 8 amino acids of a lysine, histidine, or tyrosine. In aspects “proximal” means that FSY is within 1 to 7 amino acids of a lysine, histidine, or tyrosine. In aspects “proximal” means that FSY is within 1 to 6 amino acids of a lysine, histidine, or tyrosine. In aspects “proximal” means that FSY is within 1 to 5 amino acids of a lysine, histidine, or tyrosine. In aspects “proximal” means that FSY is within 1 to 4 amino acids of a lysine, histidine, or tyrosine. In aspects “proximal” means that FSY is within 1 to 3 amino acids of a lysine, histidine, or tyrosine. In aspects “proximal” means that FSY is within 1 to 2 amino acids of a lysine, histidine, or tyrosine. In aspects “proximal” means that FSY is adjacent a lysine, histidine, or tyrosine. In aspects, FSY and the lysine, histidine, or tyrosine are in an α-strand of the protein. In aspects, FSY and the lysine, histidine, or tyrosine are in a β-strand of the protein. In aspects, the protein is a hormone. In aspects, the protein is a hormone receptor.

Provided here are biomolecule conjugates comprising a first biomolecule moiety conjugated to a second biomolecule moiety through a bioconjugate linker, wherein the bioconjugate linker has the formula:

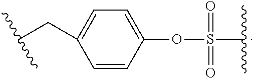 [SMILES: *Cc1ccc(OS(*)(=O)=O)cc1]

In aspects, the first biomolecule moiety and the second biomolecule moiety are each independently a peptidyl moiety. In aspects, the biomolecule conjugate is a protein conjugate. In aspects, the biomolecule conjugate is a protein conjugate, wherein the bioconjugate linker is an intramolecular linker. In aspects, the protein conjugate comprises a plurality of intramolecular linkers. In aspects, the biomolecule conjugate is a protein conjugate, wherein the bioconjugate linker is an intermolecular linker. In aspects, the protein conjugate comprises a plurality of intermolecular linkers. In aspects, the protein conjugate comprises intramolecular linkers and intermolecular linkers.

In embodiments, the biomolecule conjugate has the formula: R¹-L¹-A-X¹-L²-R²; wherein, A is the bioconjugate linker; R¹ is the first biomolecule moiety; R² is the second bioconjugate moiety; L¹ is a bond or a first covalent linker; L² is a bond of a second covalent linker; and

X¹ is —NR⁵—, —O—, —S—, or

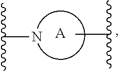

wherein ring A is a substituted or unsubstituted heteroarylene or substituted or unsubstituted heterocycloalkylene, and wherein the nitrogen in A is attached to the bioconjugate linker. R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R^{3A}, R^{3B}, R^{4A} and R^{4B} are independently hydrogen, substituted or unsubstituted alkylyl, substituted or unsubstituted heteroalkylyl, substituted or unsubstituted cycloalkylyl, substituted or unsubstituted heterocycloalkylyl, substituted or unsubstituted arylyl, or substituted or unsubstituted heteroarylyl.

In embodiments, X¹ is —NR⁵—, —O—, —S—, or

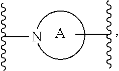

wherein ring A is a substituted or unsubstituted heteroarylene or substituted or unsubstituted heterocycloalkylene. In aspects, X¹ is —NR⁵—. In aspects X¹ is —O—. In aspects, X¹ is —S—. In aspects, X¹ is

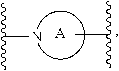

wherein ring A is a substituted or unsubstituted heteroarylene or substituted or unsubstituted heterocycloalkylene. In aspects, ring A is substituted or unsubstituted heteroarylene. In aspects, ring A is substituted or unsubstituted heterocycloalkylene. In aspects, ring A is unsubstituted heteroarylene. In aspects, ring A is unsubstituted heterocycloalkylene. In aspects, ring A is substituted heterocycloalkylene (e.g., 3 to 8 membered, 3 to 6 membered, 4 to 6 membered, 4 to 5 membered, or 5 to 6 membered). In aspects, ring A is unsubstituted heterocycloalkylene (e.g., 3 to 8 membered, 3 to 6 membered, 4 to 6 membered, 4 to 5 membered, or 5 to 6 membered). In aspects, ring A is substituted or unsubstituted heteroarylene (e.g., 5 to 10 membered, 5 to 9 membered, or 5 to 6 membered). In aspects, ring A is substituted heteroarylene (e.g., 5 to 10 membered, 5 to 9 membered, or 5 to 6 membered). In aspects, ring A is unsubstituted heteroarylene (e.g., 5 to 10 membered, 5 to 9 membered, or 5 to 6 membered).

In embodiments, R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In aspects, R⁵ is hydrogen.

In embodiments, R⁵ is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R⁵ is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₁-C₁₀, C₆-C₈, C₆-C₅) aryl or substituted or unsubstituted (e.g., 5 to 10 membered, 5 to 8 membered, 5 to 6 membered) heteroaryl.

In embodiments, R⁵ is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₁-C₁₀, C₆-C₈, C₆-C₅) aryl or unsubstituted (e.g., 5 to 10 membered, 5 to 8 membered, 5 to 6 membered) heteroaryl.

In embodiments, L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In aspects, L¹ is a bond, substituted or unsubstituted alkylene, or substituted or unsubstituted heteroalkylene. In aspects, L¹ is a bond, unsubstituted alkylene, or unsubstituted heteroalkylene. In aspects, L¹ is unsubstituted alkylene. In aspects, L¹ is unsubstituted heteroalkylene. In aspects, L¹ is a bond.

In embodiments, L¹ is-O—, —S—, R³²-substituted or unsubstituted C₁-C₂ alkylene (e.g., C₁ or C₂) or R³²-substituted or unsubstituted 2 membered heteroalkylene. In aspects, L¹ is R³²-substituted or unsubstituted alkylene (e.g., C₁-C₈ alkylene, C₁-C₆ alkylene, or C₁-C₄ alkylene), R³²-substituted or unsubstituted heteroalkylene (e.g., 2 to 8 membered heteroalkylene, 2 to 6 membered heteroalkylene, or 2 to 4 membered heteroalkylene), R³²-substituted or unsubstituted cycloalkylene (e.g., C₃-C₈ cycloalkylene, C₃-C₆ cycloalkylene, or C₅-C₆ cycloalkylene), R³²-substituted or unsubstituted heterocycloalkylene (e.g., 3 to 8 membered heterocycloalkylene, 3 to 6 membered heterocycloalkylene, or 5 to 6 membered heterocycloalkylene), R³²-substituted or unsubstituted arylene (e.g., C₆-C₁₀ arylene, C₁₀ arylene, or phenylene), or R³²-substituted or unsubstituted heteroarylene (e.g., 5 to 10 membered heteroarylene, 5 to 9 membered heteroarylene, or 5 to 6 membered heteroarylene). In aspects, L¹ is independently —O—, —S—, unsubstituted C₁-C₂ alkylene (e.g., C₁ or C₂) or unsubstituted 2 membered heteroalkylene. In aspects, L¹ is independently unsubstituted methylene. In aspects, L¹ is independently unsubstituted ethylene. In aspects, L¹ is substituted 2 membered heteroalkylene. In aspects, L¹ is substituted 3 membered heteroalkylene. In aspects, L¹ is substituted 4 membered heteroalkylene. In aspects, L¹ is an unsubstituted 2 membered heteroalkylene. In aspects, L¹ is an unsubstituted 3 membered heteroalkylene. In aspects, L¹ is an unsubstituted 4 membered heteroalkylene.

R³² is independently oxo, halogen, —CX³²₃, —CHX³²₂, —CH₂X³², —OCX³²₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³³-substituted or unsubstituted alkyl (e.g., C₁-C₈, C₁-C₆, C₁-C₄, or C₁-C₂), R³³-substituted or unsubstituted heteroalkyl (e.g., 2 to 8 membered, 2 to 6 membered, 4 to 6 membered, 2 to 3 membered, or 4 to 5 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 8 membered, 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered). In aspects, R³² is independently oxo, halogen, —CX³²₃, —CHX³²₂, —CH₂X³², —OCX³²₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³² is independently —F, —Cl, —Br, or —I.

In embodiments, R³² is independently unsubstituted methyl. In aspects, R³² is independently unsubstituted ethyl.

R³³ is independently oxo, halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁴-substituted or unsubstituted alkyl (e.g., C₁-C₈, C₁-C₆, C₁-C₄, or C₁-C₂), R³⁴-substituted or unsubstituted heteroalkyl (e.g., 2 to 8 membered, 2 to 6 membered, 4 to 6 membered, 2 to 3 membered, or 4 to 5 membered), R³⁴-substituted or unsubstituted cycloalkyl (e.g., C₃-C₈, C₃-C₆, C₄-C₆, or C₅-C₆), R³⁴-substituted or unsubstituted heterocycloalkyl (e.g., 3 to 8 membered, 3 to 6 membered, 4 to 6 membered, 4 to 5 membered, or 5 to 6 membered), R³⁴-substituted or unsubstituted aryl (e.g., C₆-C₁₀ or phenyl), or R³⁴-substituted or unsubstituted heteroaryl (e.g., 5 to 10 membered, 5 to 9 membered, or 5 to 6 membered). In aspects, R³³ is independently oxo, halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³³ is independently —F, —Cl, —Br, or —I.

In embodiments, R³³ is independently unsubstituted methyl. In aspects, R³³ is independently unsubstituted ethyl.

R³⁴ is independently oxo, halogen, —CX³⁴₃, —CHX³⁴₂, —CH₂X³⁴, —OCX³⁴₃, —OCH₂X³⁴, —OCHX³⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁴ is independently —F, —Cl, —Br, or —I.

In embodiments, R³⁴ is independently unsubstituted methyl. In aspects, R³⁴ is independently unsubstituted ethyl.

In embodiments, R^{3A} is hydrogen, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R^{3A} is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R^{3A} is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₈, C₆-C₅) aryl or substituted or unsubstituted (e.g., 5 to 10 membered, 5 to 8 membered, 5 to 6 membered,) heteroaryl.

In embodiments, R^{3A} is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₈, C₆-C₅) aryl or unsubstituted (e.g., 5 to 10 membered, 5 to 8 membered, 5 to 6 membered) heteroaryl.

In embodiments, R^{3B} is hydrogen, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R^{3B} is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R^{3B} is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₁-C₁₀, C₆-C₈, C₆-C₅) aryl or substituted or unsubstituted (e.g., 5 to 10 membered, 5 to 8 membered, 5 to 6 membered,) heteroaryl.

In embodiments, R^{3B} is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₁-C₁₀, C₆-C₈, C₆-C₅) aryl or unsubstituted (e.g., 5 to 10 membered, 5 to 8 membered, 5 to 6 membered,) heteroaryl.

In embodiments, L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L² is a bond, substituted or unsubstituted alkylene, or substituted or unsubstituted heteroalkylene. In aspects, L² is a bond, unsubstituted alkylene, or unsubstituted heteroalkylene. In aspects, L² is unsubstituted alkylene. In aspects, L² is unsubstituted heteroalkylene. In aspects, L² is a bond.

In embodiments, L² is —O—, —S—, R³⁵-substituted or unsubstituted C₁-C₂ alkylene (e.g., C₁ or C₂) or R³⁵-substituted or unsubstituted 2 membered heteroalkylene. In aspects, L² is R³⁵-substituted or unsubstituted alkylene (e.g., C₁-C₈ alkylene, C₁-C₆ alkylene, or C₁-C₄ alkylene), R³⁵-substituted or unsubstituted heteroalkylene (e.g., 2 to 8 membered heteroalkylene, 2 to 6 membered heteroalkylene, or 2 to 4 membered heteroalkylene), R³⁵-substituted or unsubstituted cycloalkylene (e.g., C₃-C₈ cycloalkylene, C₃-C₆ cycloalkylene, or C₅-C₆ cycloalkylene), R³⁵-substituted or unsubstituted heterocycloalkylene (e.g., 3 to 8 membered heterocycloalkylene, 3 to 6 membered heterocycloalkylene, or 5 to 6 membered heterocycloalkylene), R³⁵-substituted or unsubstituted arylene (e.g., C₆-C₁₀ arylene, C₁₀ arylene, or phenylene), or R³⁵-substituted or unsubstituted heteroarylene (e.g., 5 to 10 membered heteroarylene, 5 to 9 membered heteroarylene, or 5 to 6 membered heteroarylene). In aspects, L² is —O—, —S—, unsubstituted C₁-C₂ alkylene (e.g., C₁ or C₂) or unsubstituted 2 membered heteroalkylene. In aspects, L² is unsubstituted methylene. In aspects, L² is unsubstituted ethylene. In aspects, L² is substituted 2 membered heteroalkylene. In aspects, L² is substituted 3 membered heteroalkylene. In aspects, L² is substituted 4 membered heteroalkylene. In aspects, L² is an unsubstituted 2 membered heteroalkylene. In aspects, L² is an unsubstituted 3 membered heteroalkylene. In aspects, L² is an unsubstituted 4 membered heteroalkylene.

R³⁵ is independently oxo, halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁶-substituted or unsubstituted alkyl (e.g., C₁-C₈, C₁-C₆, C₁-C₄, or C₁-C₂), R³⁶-substituted or unsubstituted heteroalkyl (e.g., 2 to 8 membered, 2 to 6 membered, 4 to 6 membered, 2 to 3 membered, or 4 to 5 membered), R³⁶-substituted or unsubstituted cycloalkyl (e.g., C₃-C₈, C₃-C₆, C₄-C₆, or C₅-C₆), R³⁶-substituted or unsubstituted heterocycloalkyl (e.g., 3 to 8 membered, 3 to 6 membered, 4 to 6 membered, 4 to 5 membered, or 5 to 6 membered), R³⁶-substituted or unsubstituted aryl (e.g., C₆-C₁₀ or phenyl), or R³⁶-substituted or unsubstituted heteroaryl (e.g., 5 to 10 membered, 5 to 9 membered, or 5 to 6 membered). In aspects, R³⁵ is independently oxo, halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁵ is independently —F, —Cl, —Br, or —I.

In embodiments, R³⁵ is independently unsubstituted methyl. In aspects, R³⁵ is independently unsubstituted ethyl.

R³⁶ is independently oxo, halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, 2 to 3 membered, or 4 to 5 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 8 membered, 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered). In aspects, R³⁶ is independently oxo, halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁶ is independently —F, —Cl, —Br, or —I.

In embodiments, R³⁶ is independently unsubstituted methyl. In aspects, R³⁶ is independently unsubstituted ethyl.

R³⁷ is independently oxo, halogen, —CX³⁷₃, —CHX³⁷₂, —CH₂X³⁷, —OCX³⁷₃, —OCH₂X³⁷, —OCHX³⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁷ is independently —F, —Cl, —Br, or —I.

In embodiments, R³⁷ is independently unsubstituted methyl. In aspects, R³⁷ is independently unsubstituted ethyl.

In embodiments, R^{4A} is hydrogen, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R^{4A} is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R^{4A} is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₈, C₆—C) aryl or substituted or unsubstituted (e.g., 5 to 10 membered, 5 to 8 membered, 5 to 6 membered,) heteroaryl.

In embodiments, R^{4A} is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₈, C₆-C₅) aryl or unsubstituted (e.g., 5 to 10 membered, 5 to 8 membered, 5 to 6 membered,) heteroaryl.

In embodiments, R^{4B} is hydrogen, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, R^{4B} is hydrogen, substituted (e.g., substituted with a substituent group(s), a size-limited substituent or a lower substituent group(s)) or unsubstituted alkyl, substituted (e.g., substituted with a substituent group(s), a size-limited substituent or a lower substituent group(s)) or unsubstituted heteroalkyl, substituted (e.g., substituted with a substituent group(s), a size-limited substituent or a lower substituent group(s)) or unsubstituted cycloalkyl, substituted (e.g., substituted with a substituent group(s), a size-limited substituent or a lower substituent group(s)) or unsubstituted heterocycloalkyl, substituted (e.g., substituted with a substituent group(s), a size-limited substituent or a lower substituent group(s)) or unsubstituted aryl or substituted (e.g., substituted with a substituent group(s), a size-limited substituent or a lower substituent group(s)) or unsubstituted heteroaryl.

In embodiments, R^{4B} is hydrogen, substituted or unsubstituted (e.g., C₁-C₂₀, C₁-C₁₀, C₁-C₅) alkyl, substituted or unsubstituted (e.g., 2 to 20 membered, 2 to 10 membered, 2 to 5 membered) heteroalkyl, substituted or unsubstituted (e.g., C₃-C₈, C₃-C₆, C₃-C₅) cycloalkyl, substituted or unsubstituted (e.g., 3 to 8 membered, 3 to 6 membered, 3 to 5 membered) heterocycloalkyl, substituted or unsubstituted (e.g., C₆-C₁₀, C₆-C₈, C₆-C₅) aryl or substituted or unsubstituted (e.g., 5 to 10 membered, 5 to 8 membered, 5 to 6 membered,) heteroaryl.

In embodiments, R^{4B} is hydrogen, unsubstituted (e.g., C₁-C₂₀, C₁-C₁₀, C₁-C₅) alkyl, unsubstituted (e.g., 2 to 20 membered, 2 to 10 membered, 2 to 5 membered) heteroalkyl, unsubstituted (e.g., C₃-C₈, C₃-C₆, C₃-C₅) cycloalkyl, unsubstituted (e.g., 3 to 8 membered, 3 to 6 membered, 3 to 5 membered) heterocycloalkyl, unsubstituted (e.g., C₆-C₁₀, C₆-C₈, C₆-C₅) aryl or unsubstituted (e.g., 5 to 10 membered, 5 to 8 membered, 5 to 6 membered,) heteroaryl.

In embodiments, X¹ is imidazolylene, —NH— or —O—. In aspects, X¹ is imidazolylene (i.e., a divalent imidazole). In aspects, X¹ is —NH—. In aspects, X¹ is —O—.

In embodiments, the first biomolecule moiety is a peptidyl moiety. In aspects, the second biomolecule moiety is a peptidyl moiety. In aspects, the first biomolecule moiety is a peptidyl moiety and the second biomolecule moiety is a peptidyl moiety. In aspects, the peptidyl moieties in the first biomolecule moiety and the second biomolecule moiety are in the same protein. In aspects, the peptidyl moieties in the first biomolecule moiety and the second biomolecule moiety are in different proteins.

In embodiments, -L¹-R¹ is a peptidyl moiety. In embodiments, -L²-R² is a peptidyl moiety. In aspects, the peptidyl moieties of -L¹-R¹ and -L²-R² are in the same protein. In aspects, the peptidyl moieties of -L¹-R¹ and -L²-R² are in different proteins.

In embodiments, the first biomolecule moiety is a nucleic acid moiety or a carbohydrate moiety. In embodiments, the first biomolecule moiety is a nucleic acid moiety. In embodiments, the first biomolecule moiety is a carbohydrate moiety. In embodiments, the second biomolecule moiety is a nucleic acid moiety or a carbohydrate moiety. In embodiments, the second biomolecule moiety is a nucleic acid moiety. In embodiments, the second biomolecule moiety is a carbohydrate moiety.

In embodiments, -L¹-R¹ is a nucleic acid moiety or a carbohydrate moiety. In aspects, -L¹-R¹ is a nucleic acid moiety. In aspects, -L¹-R¹ is a carbohydrate moiety. In aspects, -L²-R² is a nucleic acid moiety or a carbohydrate moiety. In aspects, -L²-R² is a nucleic acid moiety. In aspects, -L²-R² is a carbohydrate moiety.

In embodiments, the first biomolecule moiety is selected from the group consisting of a peptidyl moiety, a nucleic acid moiety, and a carbohydrate moiety. In aspects, the second biomolecule moiety is selected from the group consisting of a peptidyl moiety, a nucleic acid moiety, and a carbohydrate moiety. In aspects, the first biomolecule moiety is same as the second biomolecule moiety. In aspects, the first biomolecule moiety is different from the second biomolecule moiety. In aspects, the first biomolecule moiety and the second biomolecule moiety are within the same biomolecule. In aspects, the first biomolecule moiety and the second biomolecule moiety are in different biomolecules. In aspects, the first biomolecule moiety and the second biomolecule moiety are each independently a peptidyl moiety.

In embodiments, -L¹-R¹ is selected from the group consisting of a peptidyl moiety, a nucleic acid moiety and a carbohydrate moiety. In aspects, -L²-R² is selected from the group consisting of a peptidyl moiety, a nucleic acid moiety and a carbohydrate moiety. In aspects, -L¹-R¹ is the same as -L²-R². In aspects, -L¹-R¹ is different from -L²-R². In aspects, -L¹-R¹ and -L²-R² are each independently a peptidyl moiety.

In aspects, the disclosure provides a protein comprising a moiety of Formula (A), a moiety of Formula (B), a moiety of Formula (C), or a combination of two or more thereof:

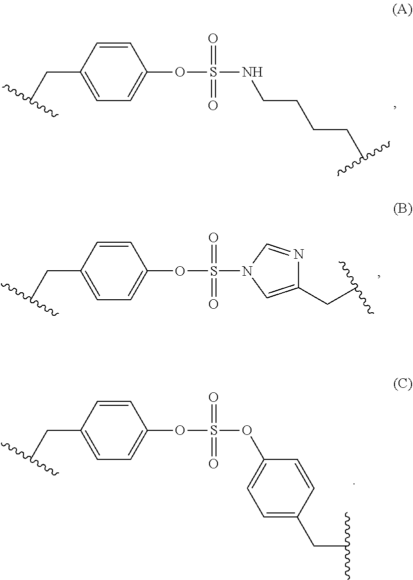 [SMILES: *CCCCNS(=O)(=O)Oc1ccc(C*)cc1 ; *Cc1ccc(OS(=O)(=O)n2cnc(C*)c2)cc1 ; *Cc1ccc(OS(=O)(=O)Oc2ccc(C*)cc2)cc1]

In aspects, the protein comprises a moiety of Formula (A). In aspects, the protein comprises a moiety of Formula (B). In aspects, the protein comprises a moiety of Formula (C). In aspects, the protein comprises a moiety of Formula (A) and a moiety of Formula (B). In aspects, the protein comprises a moiety of Formula (A) and a moiety of Formula (C). In aspects, the protein comprises a moiety of Formula (B) and a moiety of Formula (C). In aspects, the protein comprises a moiety of Formula (A), a moiety of Formula (B), and a moiety of Formula (C). In aspect, the moieties of Formula (A), (B), (C), or a combination thereof, form intramolecular covalent bonds. In aspect, the moiety of Formula (A) forms an intramolecular covalent bond. In aspect, the moiety of Formula (B) forms an intramolecular covalent bond. In aspect, the moiety of Formula (C) forms an intramolecular covalent bond. In aspect, the moieties of Formula (A) and (B) form intramolecular covalent bonds. In aspect, the moieties of Formula (A) and (C) form intramolecular covalent bonds. In aspect, the moieties of Formula (B) and (C) form intramolecular covalent bonds. In aspect, the moieties of Formula (A), (B), and (C) form intramolecular covalent bonds. In aspect, the moieties of Formula (A), (B), (C), or a combination thereof form intermolecular covalent bonds. In aspect, the moiety of Formula (A) forms an intermolecular covalent bond. In aspect, the moiety of Formula (B) forms an intermolecular covalent bond. In aspects, the moiety of Formula (C) forms an intermolecular covalent bond. In aspect, the moieties of Formula (A) and (B) form intermolecular covalent bonds. In aspect, the moieties of Formula (A) and (C) form intermolecular covalent bonds. In aspect, the moieties of Formula (B) and (C) form intermolecular covalent bonds. In aspect, the moieties of Formula (A), (B), and (C) form intermolecular covalent bonds.

In aspects, the disclosure provides a protein of Formula (I), Formula (II), or Formula (III):

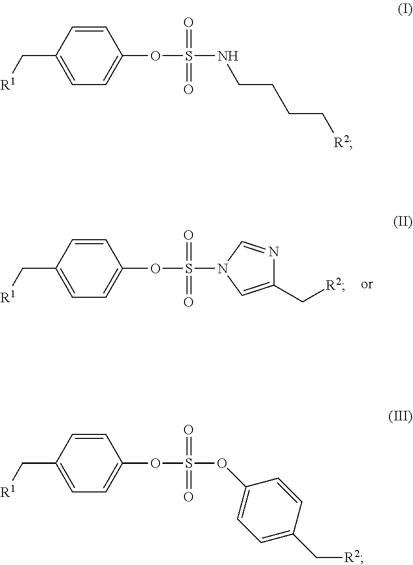

wherein R¹ and R² are each independently a peptidyl moiety that are joined together, i.e., the protein of Formula (I), (II), and (III) comprises an intramolecular covalent bond. In aspects, the protein is Formula (I). In aspects, the protein is Formula (II). In aspects, the protein is Formula (III). In aspects, the peptidyl moiety of R¹ and the peptidyl moiety of R² comprise a protein α-strand. In aspects, the peptidyl moiety of R¹ and the peptidyl moiety of R² comprise a protein β-strand. In aspects, the peptidyl moiety of R¹ comprises a protein α-strand and the peptidyl moiety of R² comprises a protein β-strand. In aspects, the peptidyl moiety of R¹ comprises a protein β-strand and the peptidyl moiety of R² comprises a protein α-strand.

In aspects, the disclosure provides a protein of Formula (I), Formula (II), or Formula (III):

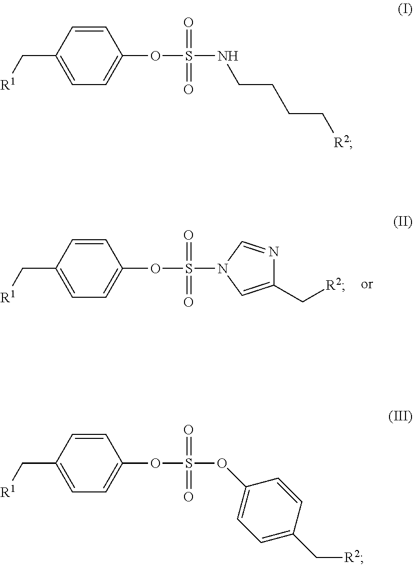

wherein R¹ is a peptidyl moiety of a first protein and R² is a peptidyl moiety of a second protein, i.e., there is an intermolecular covalent bond between two proteins. In aspects, the intermolecular bond is between two different proteins. In aspects, the intermolecular bond is between two of the same proteins (e.g., two proteins having the same amino acid sequence that are intermolecularly bonded). In aspects the first protein is covalently bonded to the second protein via the moiety of Formula (A) to form an intermolecularly bonded protein of Formula (I). In aspects the first protein is covalently bonded to the second protein via the moiety of Formula (B) to form an intermolecularly bonded protein of Formula (II). In aspects the first protein is covalently bonded to the second protein via the moiety of Formula (C) to form an intermolecularly bonded protein of Formula (III). In aspects the first protein is covalently bonded to the second protein via the moiety of Formula (A) and the moiety of Formula (A). In aspects the first protein is covalently bonded to the second protein via the moiety of Formula (A) and the moiety of Formula (C). In aspects the first protein is covalently bonded to the second protein via the moiety of Formula (B) and the moiety of Formula (C). In aspects the first protein is covalently bonded to the second protein via the moiety of Formula (A), the moiety of Formula (B), and the moiety of Formula (C). In aspects, the first protein is a hormone and the second protein is the receptor for the hormone. In aspects, the peptidyl moiety R¹ and R² comprise a protein α-strand. In aspects, the peptidyl moiety R¹ and R² comprise a protein β-strand. In aspects, the peptidyl moiety R¹ comprises a protein α-strand and the peptidyl moiety R² comprises a protein β-strand. In aspects, the peptidyl moiety R¹ comprises a protein β-strand and the peptidyl moiety R² comprises a protein α-strand.

In aspects, the protein conjugates may comprise three or more different and/or separate proteins. For example, the first protein is covalently bonded to the second protein via a moiety of Formula (A), a moiety of Formula (B), a moiety of Formula (C), or a combination of two or more thereof, and the second protein is covalently bonded to a third protein via a moiety of Formula (A), a moiety of Formula (B), a moiety of Formula (C), or a combination of two or more thereof. As another example, the first protein is covalently bonded to the second protein via a moiety of Formula (A), a moiety of Formula (B), a moiety of Formula (C), or a combination of two or more thereof, and the first protein is also covalently bonded to a third protein via a moiety of Formula (A), a moiety of Formula (B), a moiety of Formula (C), or a combination of two or more thereof. In each of these aspects, the first protein, the second protein, and the third protein may each optionally further comprise a moiety of Formula (A), a moiety of Formula (B), a moiety of Formula (C), or a combination of two or more thereof, wherein the peptidyl moiety of R¹ and R² form intramolecular bonds within the first protein, the second protein, or the third protein, respectively.

Pyrrolysyl-tRNA Synthetase

As described herein, an unnatural amino acid (e.g., FSY) may be inserted into or replace a naturally occurring amino acid in a biomolecule (e.g., protein). In order for the unnatural amino acid to be inserted or replace an amino acid in a biomolecule (e.g., protein), it must be capable of being incorporated during proteinogenesis. Thus, the unnatural amino acid must be present on a transfer RNA molecule (tRNA) such that it may be used in translation. Loading of amino acids occurs via an aminoacyl-tRNA synthetase, which is an enzyme that facilitates the attachment of appropriate amino acids to tRNA molecules. However, the attachment of unnatural amino acids to tRNA may not necessarily be accomplished by the naturally occurring aminoacyl-tRNA synthetase. Engineered aminoacyl-tRNA synthetases (e.g. mutant pyrrolysyl-tRNA synthetase (PyIRS)) may be useful for attaching unnatural amino acids to tRNA. A PyIRS mutant library was generated. Compared to previously described PyIRS mutant library, the PylRS mutant library generated herein was constructed using the new small-intelligent mutagenesis approach that allows a greater number of amino acid residues to be mutated simultaneously (e.g., 10 amino acid residues). Out of 2.76×10⁷ clones selected and screened in total, one PyIRS mutant (in 6 clones) was identified that is capable of attaching FSY (see, e.g., Example 1).

The disclosure provides a mutant pyrrolysyl-tRNA synthetase, including at least 5 amino acid residues substitutions within the substrate-binding site of the mutant pyrrolysyl-tRNA synthetase. In aspects, the mutant pyrrolysyl-tRNA synthetase comprises at least 5 amino acid residues substitutions in the amino acid sequence of SEQ ID NO:3. In aspects, the substrate-binding site includes residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO:3. In aspects, the at least 5 amino acid residues substitutions are a substitution for alanine at position 302, a substitution for asparagine at position 346, a substitution for cysteine at position 348, a substitution for tyrosine at position 384, and a substitution for tryptophan at position 417 as set forth in the amino acid sequence of SEQ ID NO:3. In aspects, the at least 5 amino acid residues substitutions are isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO:3.

In embodiments, the mutant pyrrolysyl-tRNA synthetase has the amino acid sequence of SEQ ID NO:1. In aspects, the mutant pyrrolysyl-tRNA synthetase includes an amino acid sequence of SEQ ID NO:1. In aspects, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO:1. In aspects, the mutant pyrrolysyl-tRNA synthetase has an amino acid sequence that has at least 80% identity to SEQ ID NO:1. In aspects, the mutant pyrrolysyl-tRNA synthetase has an amino acid sequence that has at least 85% identity to SEQ ID NO:1. In aspects, the mutant pyrrolysyl-tRNA synthetase has an amino acid sequence that has at least 90% identity to SEQ ID NO:1. In aspects, the mutant pyrrolysyl-tRNA synthetase has an amino acid sequence that has at least 95% identity to SEQ ID NO:1. In aspects, the mutant pyrrolysyl-tRNA synthetase has an amino acid sequence that has at least 98% identity to SEQ ID NO:1.

In embodiments, the mutant pyrrolysyl-tRNA synthetase is encoded by the nucleic acid sequence of SEQ ID NO:2. In aspects, the mutant pyrrolysyl-tRNA synthetase is encoded by a nucleic acid sequence including the sequence of SEQ ID NO:2. In aspects, the mutant pyrrolysyl-tRNA synthetase is encoded by a nucleic acid sequence that is at least 80%, 85%, 90%, 91%, 92%, 93%, 94%, 95%, 96%, 97%, 98%, 99%, or 100% identity to SEQ ID NO: 2. In aspects, the mutant pyrrolysyl-tRNA synthetase has an amino acid sequence that has at least 80% identity to SEQ ID NO:2. In aspects, the mutant pyrrolysyl-tRNA synthetase has an amino acid sequence that has at least 85% identity to SEQ ID NO:2. In aspects, the mutant pyrrolysyl-tRNA synthetase has an amino acid sequence that has at least 90% identity to SEQ ID NO:2. In aspects, the mutant pyrrolysyl-tRNA synthetase has an amino acid sequence that has at least 95% identity to SEQ ID NO:2. In aspects, the mutant pyrrolysyl-tRNA synthetase has an amino acid sequence that has at least 98% identity to SEQ ID NO:2.

Vectors

It is contemplated that the compositions (e.g., mutant pyrrolysyl-tRNA synthetase, tRNA^{Pyl}) provided herein may be delivered to cells using methods well known in the art. Thus, in an aspect is provided a vector including a nucleic acid sequence encoding a mutant pyrrolysyl-tRNA synthetase as described herein, including embodiments thereof. In aspects, the vector comprises a nucleic acid sequence encoding a mutant pyrrolysyl-tRNA synthetase that comprises at least 5 amino acid residues substitutions within the substrate-binding site of the mutant pyrrolysyl-tRNA synthetase. In aspects, the vector further includes a nucleic acid sequence encoding tRNA^{Pyl}. In aspects, the vector comprises a nucleic acid sequence encoding a mutant pyrrolysyl-tRNA synthetase that comprises at least 5 amino acid residues substitutions in the amino acid sequence of SEQ ID NO:3. In aspects, the vector further includes a nucleic acid sequence encoding tRNA^{Pyl}. In aspects, the vector comprises a nucleic acid sequence encoding a mutant pyrrolysyl-tRNA synthetase that comprises amino acid substitutions of residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO:3. In aspects, the vector further includes a nucleic acid sequence encoding tRNA^{Pyl}. In aspects, the vector comprises a nucleic acid sequence encoding a mutant pyrrolysyl-tRNA synthetase that comprises amino acid substitutions of residues alanine at position 302, a substitution for asparagine at position 346, a substitution for cysteine at position 348, a substitution for tyrosine at position 384, and a substitution for tryptophan at position 417 as set forth in the amino acid sequence of SEQ ID NO:3. In aspects, the vector further includes a nucleic acid sequence encoding tRNA^{Pyl}. In aspects, the vector comprises a nucleic acid sequence encoding a mutant pyrrolysyl-tRNA synthetase that comprises amino acid substitutions of residues isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO:3. In aspects, the vector further includes a nucleic acid sequence encoding tRNA^{Pyl}.

In embodiments, the nucleic acid sequence encoding tRNA^{Pyl} is the sequence set forth in SEQ ID NO:4. In aspects, the nucleic acid sequence encoding tRNA^{Pyl} comprises the sequence set forth in SEQ ID NO:4. In aspects, the nucleic acid sequence encoding tRNA^{Pyl} has a sequence that has at least 80%, 85%, 90%, 91%, 92%, 93%, 94%, 95%, 96%, 97%, 98%, 99%, or 100% identity to SEQ ID NO:4. In aspects, the nucleic acid sequence encoding tRNA^{Pyl} has a sequence that has at least 80%, identity to SEQ ID NO:4. In aspects, the nucleic acid sequence encoding tRNA^{Pyl} has a sequence that has at least 85%, identity to SEQ ID NO:4. In aspects, the nucleic acid sequence encoding tRNA^{Pyl} has a sequence that has at least 90%, identity to SEQ ID NO:4. In aspects, the nucleic acid sequence encoding tRNA^{Pyl} has a sequence that has at least 95%, identity to SEQ ID NO: 4. In aspects, the nucleic acid sequence encoding tRNA^{Pyl} has a sequence that has at least 98%, identity to SEQ ID NO:4.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. The terms “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Exemplary vectors that can be used include, but are not limited to, pEvol vector, pMP vector, pET vector, pTak vector, pBad vector (see, e.g., Example 1).

Complexes

In an aspect is provided a complex including a mutant pyrrolysyl-tRNA synthetase as described herein, including embodiments thereof, and fluorosulfate-L-tyrosine (FSY) having the following formula:

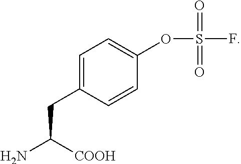 [SMILES: N[C@@H](Cc1ccc(OS(=O)(=O)F)cc1)C(=O)O]

In aspects, the complex comprises a mutant pyrrolysyl-tRNA synthetase that comprises at least 5 amino acid residues substitutions within the substrate-binding site of the pyrrolysyl-tRNA synthetase of SEQ ID NO:3. In aspects, the mutant pyrrolysyl-tRNA synthetase comprises amino acid residue substitutions within the substrate-binding site at residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO:3. In aspects, the mutant pyrrolysyl-tRNA synthetase comprises amino acid residue substitutions within the substrate-binding site at residues alanine at position 302, a substitution for asparagine at position 346, a substitution for cysteine at position 348, a substitution for tyrosine at position 384, and a substitution for tryptophan at position 417 as set forth in the amino acid sequence of SEQ ID NO:3. In aspects, the at least 5 amino acid residues substitutions are isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO:3. In aspects, the mutant pyrrolysyl-tRNA comprises the amino acid sequence of SEQ ID NO:1. In aspects, the mutant pyrrolysyl-tRNA has at least 80% sequence identity to the amino acid sequence of SEQ ID NO:1. In aspects, the mutant pyrrolysyl-tRNA has at least 85% sequence identity to the amino acid sequence of SEQ ID NO:1. In aspects, the mutant pyrrolysyl-tRNA has at least 90% sequence identity to the amino acid sequence of SEQ ID NO:1. In aspects, the mutant pyrrolysyl-tRNA has at least 95% sequence identity to the amino acid sequence of SEQ ID NO:1. In aspects, the mutant pyrrolysyl-tRNA comprises the amino acid sequence of SEQ ID NO:2. In aspects, the mutant pyrrolysyl-tRNA has at least 80% sequence identity to the amino acid sequence of SEQ ID NO:2. In aspects, the mutant pyrrolysyl-tRNA has at least 85% sequence identity to the amino acid sequence of SEQ ID NO:2. In aspects, the mutant pyrrolysyl-tRNA has at least 90% sequence identity to the amino acid sequence of SEQ ID NO:2. In aspects, the mutant pyrrolysyl-tRNA has at least 95% sequence identity to the amino acid sequence of SEQ ID NO:2.

In embodiments, the complex comprises a mutant pyrrolysyl-tRNA synthetase as described herein, including embodiments thereof, fluorosulfate-L-tyrosine (FSY); and tRNA^{Pyl} as described herein, including embodiments thereof. In aspects, the tRNA^{Pyl} comprises the amino acid sequence of SEQ ID NO:4. In aspects, the tRNA^{Pyl} has at least 80% sequence identity to the amino acid sequence of SEQ ID NO:4. In aspects, the tRNA^{Pyl} has at least 85% sequence identity to the amino acid sequence of SEQ ID NO:4. In aspects, the tRNA^{Pyl} has at least 90% sequence identity to the amino acid sequence of SEQ ID NO:4. In aspects, the tRNA^{Pyl} has at least 95% sequence identity to the amino acid sequence of SEQ ID NO:4.

Cellular Compositions

The disclosure provides cells comprising the compositions and complexes provided herein, including embodiments thereof. Therefore, in an aspect is provided a cell including fluorosulfate-L-tyrosine (FSY) having the following formula:

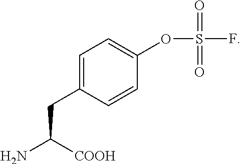 [SMILES: N[C@@H](Cc1ccc(OS(=O)(=O)F)cc1)C(=O)O]

In embodiments, the cell further includes a mutant pyrrolysyl-tRNA synthetase as described herein, including aspects thereof. In aspects, the cell further includes a vector as described herein, including aspects thereof. In aspects, the cell further includes a tRNA¹.

In embodiments, FSY is biosynthesized inside the cell, thereby generating a cell containing FSY. In aspects, FSY is contained in the medium outside the cell and penetrates into the cell, thereby generating a cell containing FSY. In aspects, the cell comprises an FSY biomolecule. In aspects, the cell comprises an FSY protein. In aspects, the cell comprises an FSY biomolecule that is synthesized inside the cell. In aspects, the cell comprises an FSY protein that is synthesized inside the cell. In aspects, the cell comprises an FSY biomolecule that is synthesized outside a cell, and that penetrates into the cell. In aspects, the cell comprises an FSY protein that is synthesized outside a cell, and that penetrates into the cell.

In embodiments, the cell comprises the biomolecule conjugates described herein. In aspects, the cell comprises biomolecule conjugate comprising a first biomolecule moiety conjugated to a second biomolecule moiety through a bioconjugate linker, wherein the bioconjugate linker has the formula:

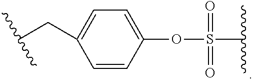 [SMILES: *Cc1ccc(OS(*)(=O)=O)cc1]

In aspects, the cell comprises a biomolecule conjugate of the formula R¹-L¹-A-X¹-L²-R², wherein the substituents are as defined herein. In aspects, the first and second biomolecule moieties are each independently a peptidyl moiety, a nucleic acid moiety, or a carbohydrate moiety. In aspects, the first and second biomolecule moieties are each a peptidyl moiety within the same protein. In aspects, the first and second biomolecule moieties are each a peptidyl moiety within different proteins.

In embodiments, the cell comprises a protein which comprises a moiety of Formula (A), a moiety of Formula (B), a moiety of Formula (C), or a combination of two or more thereof:

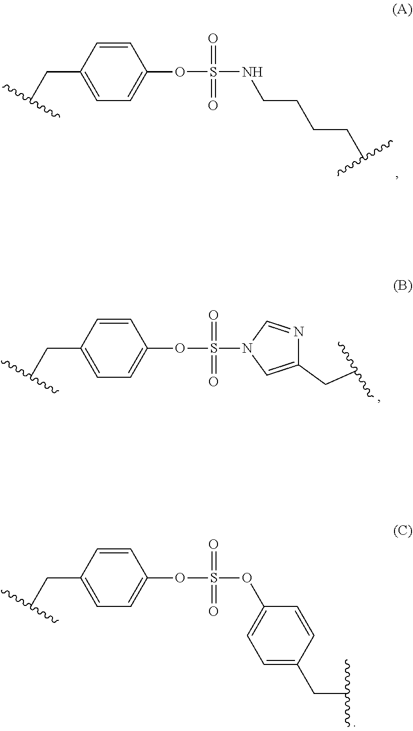 [SMILES: *CCCCNS(=O)(=O)Oc1ccc(C*)cc1 ; *Cc1ccc(OS(=O)(=O)n2cnc(C*)c2)cc1 ; *Cc1ccc(OS(=O)(=O)Oc2ccc(C*)cc2)cc1]

In aspects, the moiety of Formula (A), (B), or (C) forms an intramolecular covalent bond within a protein. In aspects, the moiety of Formula (A), (B), or (C) forms an intermolecular covalent bond between two proteins.

In embodiments, the cell comprises a protein of Formula (I), Formula (II), or Formula (III):

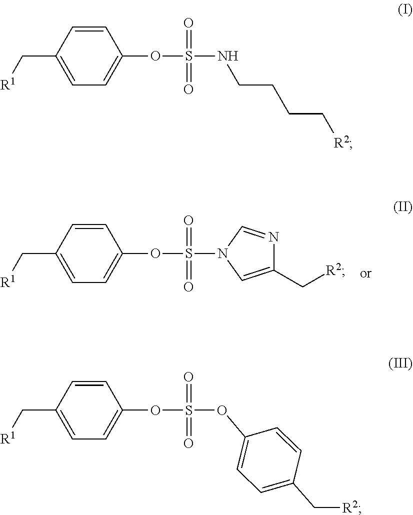

wherein R¹ and R² are each independently a peptidyl moiety. In aspects, R¹ and R² are bonded together, such that protein of Formula (I), (II), and (III) comprise an intramolecular bond. In aspects, R¹ and R² are a peptidyl moiety in two different proteins, such that the protein of Formula (I), (II), and (III) comprises an intermolecular bond between two proteins.

A cell can be any prokaryotic or eukaryotic cell. For example, any of the compositions described herein can be expressed in bacterial cells such as E. coli, insect cells, yeast or mammalian cells (such as Hela cells, Chinese hamster ovary cells (CHO) or COS cells). In aspects, the cell is a bacterial cell. In aspects, a cell can be a premature mammalian cell, i.e., pluripotent stem cell. In aspects, a cell can be derived from other human tissue. In aspects, the cell is a mammalian cell. Other suitable cells are known to those skilled in the art.

### Methods of Forming a Biomolecule or Biomolecule Conjugate

The compositions provided herein are useful for forming a biomolecule or biomolecule conjugate. Thus, in an aspect is provided method of forming an FSY biomolecule by contacting a biomolecule, a mutant pyrrolysyl-tRNA synthetase, a tRNA^{Pyl}, and fluorosulfate-L-tyrosine (FSY) having the formula:

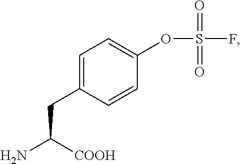 [SMILES: N[C@@H](Cc1ccc(OS(=O)(=O)F)cc1)C(=O)O]

thereby producing the FSY biomolecule, i.e., a biomolecule comprising the unnatural amino acid of FSY. The biomolecule produced by the method will comprise the unnatural amino acid side chain of the formula:

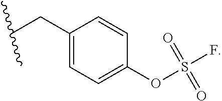 [SMILES: *Cc1ccc(OS(=O)(=O)F)cc1]

The mutant pyrrolysyl-tRNA synthetase used in the method of producing the biomolecule is any described herein. The tRNA^{Pyl} used in the method of producing the biomolecule is any described herein. In aspects, the biomolecule is a protein. In aspects, the biomolecule is a nucleic acid. In aspects, the biomolecule is a carbohydrate. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

In embodiments, the disclosure provides methods for producing an FSY protein by contacting a protein, a mutant pyrrolysyl-tRNA synthetase, a tRNA^{Pyl}, and fluorosulfate-L-tyrosine (FSY) having the formula:

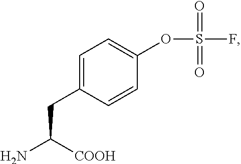 [SMILES: N[C@@H](Cc1ccc(OS(=O)(=O)F)cc1)C(=O)O]

thereby producing the FSY protein, i.e., a protein comprising the unnatural amino acid of FSY. The protein produced by the method will comprise the unnatural amino acid side chain of the formula:

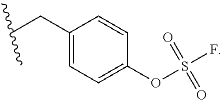 [SMILES: *Cc1ccc(OS(=O)(=O)F)cc1]

The mutant pyrrolysyl-tRNA synthetase used in the method of producing the protein is any described herein. The tRNA^{Pyl} used in the method of producing the protein is any described herein. In aspects, the FSY protein further comprises lysine, histidine, tyrosine, or two or more thereof. In aspects, the FSY protein comprises FSY that is proximal to lysine, histidine, tyrosine, or two or more thereof. In aspects, the FSY protein comprises FSY that is proximal to lysine. In aspects, the FSY protein comprises FSY that is proximal to histidine. In aspects, the FSY protein comprises FSY that is proximal to tyrosine. The term “proximal” is described herein. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

In embodiments, the disclosure provides proteins comprising one or more intramolecular covalent bonds (e.g., a protein conjugate). In aspects, FSY and the proximal lysine, histidine, or tyrosine undergo a reaction to form the intramolecular covalent bond, resulting in a moiety of Formula (A), a moiety of Formula (B), a moiety of Formula (C), or a combination of two or more thereof:

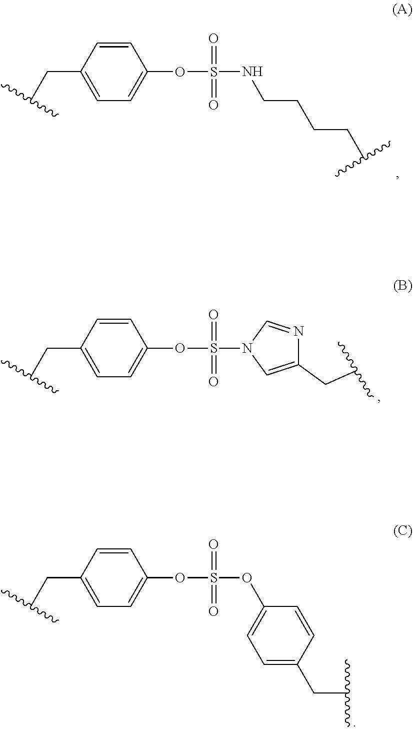 [SMILES: *CCCCNS(=O)(=O)Oc1ccc(C*)cc1 ; *Cc1ccc(OS(=O)(=O)n2cnc(C*)c2)cc1 ; *Cc1ccc(OS(=O)(=O)Oc2ccc(C*)cc2)cc1]

The FSY and the lysine, histidine, or tyrosine that are proximal thereto can be on an α-strand of the protein and/or a β-strand of the protein. In aspects, the reaction to form the intramolecular covalent bond between FSY and the lysine, histidine, or tyrosine is accomplished through click chemistry. In aspects, the reaction to form the intramolecular covalent bond between FSY and the lysine, histidine, or tyrosine is accomplished through proximity-enabled, click chemistry. In aspects, the reaction to form the intramolecular covalent bond between FSY and the lysine, histidine, or tyrosine is accomplished through a sulfur-fluoride exchange reaction. In aspects, the reaction to form the intramolecular covalent bond between FSY and the lysine, histidine, or tyrosine is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

In embodiments, the disclosure provides protein conjugates of Formula (I), (II), or (III) wherein R¹ and R² are each independently a peptidyl moiety:

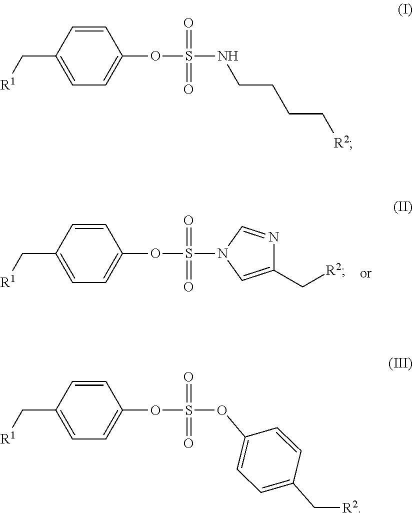

In aspects, R¹ and R² are joined together to form an intramolecularly conjugated protein. In aspects, R¹ and R² are not joined together. In aspects, the reaction to form the protein conjugates is accomplished through click chemistry. In aspects, the reaction to form the protein conjugate is accomplished through proximity-enabled, click chemistry. In aspects, the reaction to form the protein conjugate is accomplished through a sulfur-fluoride exchange reaction. In aspects, the reaction to form the protein conjugate is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

In embodiments, two or more proteins can be covalently linked by the methods and compositions described herein. In aspects, FSY is an unnatural amino acid in a first protein and lysine, histidine, or tyrosine are amino acids in a second protein, wherein the first protein and the second protein are different. The FSY in the first protein undergoes a reaction with the lysine, histidine, or tyrosine in the second protein to form an intermolecular covalent bond between the first and second proteins. The intermolecular covalent bond linking the two proteins is represented by a moiety of Formula (A), moiety of Formula (B), moiety of Formula (C), or a combination of two or more thereof:

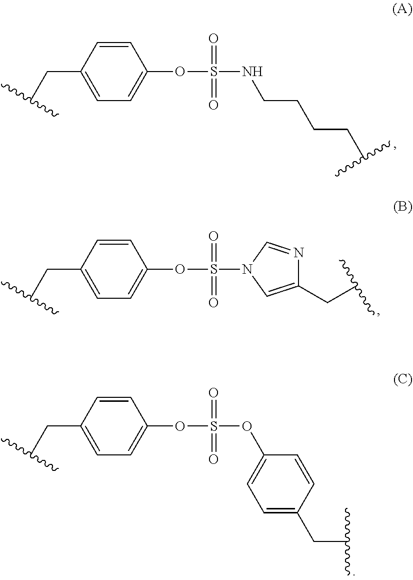 [SMILES: *CCCCNS(=O)(=O)Oc1ccc(C*)cc1 ; *Cc1ccc(OS(=O)(=O)n2cnc(C*)c2)cc1 ; *Cc1ccc(OS(=O)(=O)Oc2ccc(C*)cc2)cc1]

The FSY and the lysine, histidine, or tyrosine can be on an α-strand of their respective proteins and/or a β-strand of their respective proteins. In aspects, the reaction to form the intermolecular covalent bond between FSY in the first protein and the lysine, histidine, or tyrosine in the second protein is accomplished through click chemistry. In aspects, the reaction to form the intermolecular covalent bond between FSY in the first protein and the lysine, histidine, or tyrosine in the second protein is accomplished through proximity-enabled, click chemistry. In aspects, the reaction to form the intermolecular covalent bond between FSY in the first protein and the lysine, histidine, or tyrosine in the second protein is accomplished through sulfur-fluoride exchange. In aspects, the reaction to form the intermolecular covalent bond between FSY in the first protein and the lysine, histidine, or tyrosine in the second protein is accomplished through proximity-enabled, sulfur-fluoride exchange. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

In embodiments, the disclosure provides biomolecule conjugates comprising a first biomolecule moiety conjugated to a second biomolecule moiety through a bioconjugate linker, wherein the bioconjugate linker has the formula:

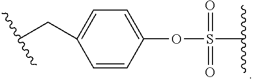 [SMILES: *Cc1ccc(OS(*)(=O)=O)cc1]

In aspects, the biomolecule conjugate has the formula R¹-L¹-A-X¹-L²-R², where the substituents are as defined herein. In aspects, the reaction to form the biomolecule conjugates is accomplished through click chemistry. In aspects, the reaction to form the biomolecule conjugate is accomplished through proximity-enabled, click chemistry. In aspects, the reaction to form the biomolecule conjugate is accomplished through a sulfur-fluoride exchange reaction. In aspects, the reaction to form the biomolecule conjugate is accomplished through a proximity-enabled, sulfur-fluoride exchange reaction. In aspects, the reaction is performed in vitro. In aspects, the reaction is performed in vivo. In aspects, the reaction is performed in one or more living cells. In aspects, the reaction is performed in one or more living bacterial cells. In aspects, the reaction is performed in one or more living mammalian cells.

### Embodiments 1-53

### Embodiment 1

A biomolecule conjugate comprising a first biomolecule moiety conjugated to a second biomolecule moiety through a bioconjugate linker, wherein the bioconjugate linker has the formula:

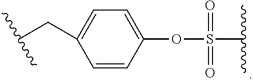 [SMILES: *Cc1ccc(OS(*)(=O)=O)cc1]

### Embodiment 2

The biomolecule conjugate of Embodiment 1, wherein the biomolecule conjugate has the formula: R¹-L¹-A-X¹-L²-R²; wherein: A is the bioconjugate linker; R¹ is the first biomolecule moiety; R² is the second biomolecule moiety; L¹ is a bond or a first covalent linker; L² is a bond or a second covalent linker; and X¹ is —NR⁵—, —O—, —S—, or

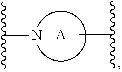

wherein ring A is a substituted or unsubstituted heteroarylene or substituted or unsubstituted heterocycloalkylene, and wherein the nitrogen in A is attached to the bioconjugate linker; and R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein R¹ and R² are optionally joined together to form an intramolecularly conjugated biomolecule conjugate.

### Embodiment 3

The biomolecule conjugate of Embodiment 2, wherein L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{3A}, R^{3B}R^{4A}, and R^{4B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 4

The biomolecule conjugate of Embodiment 2 or 3, wherein X¹ is —NH—, —O—, or imidazolylene.

### Embodiment 5

The biomolecule conjugate of any one of Embodiments 1 to 4, wherein the first biomolecule moiety is a peptidyl moiety, a nucleic acid moiety, or a carbohydrate moiety.

### Embodiment 6

The biomolecule conjugate of Embodiment 5, wherein the first biomolecule moiety is a peptidyl moiety; and wherein the peptidyl moiety is covalently bonded to the bioconjugate linker via lysine, histidine, or tyrosine.

### Embodiment 7

The biomolecule conjugate of any one of Embodiments 1 to 6, wherein the second biomolecule moiety is a peptidyl moiety, a nucleic acid moiety, or a carbohydrate moiety.

### Embodiment 8

The biomolecule conjugate of Embodiment 7, wherein the second biomolecule moiety is a peptidyl moiety; and wherein the peptidyl moiety is covalently bonded to the bioconjugate linker via lysine, histidine, or tyrosine.

### Embodiment 9

The biomolecule conjugate of Embodiment 2 or 3, wherein -L¹-R¹ is a peptidyl moiety, a nucleic acid moiety, or a carbohydrate moiety.

### Embodiment 10

The biomolecule conjugate of Embodiment 2, 3, or 9, wherein -L²-R² is a peptidyl moiety, a nucleic acid moiety, or a carbohydrate moiety.

### Embodiment 11

The biomolecule conjugate of anyone of Embodiments 1 to 10, wherein the bioconjugate linker is an intermolecular linker.

### Embodiment 12

The biomolecule conjugate of anyone of Embodiments 1 to 10, wherein the bioconjugate linker is an intramolecular linker.

### Embodiment 13

A protein of Formula (I), Formula (II), or Formula (III):

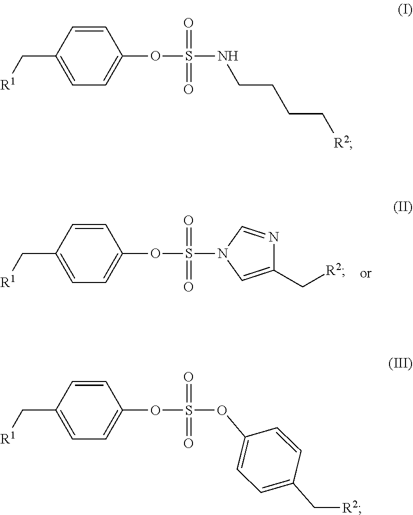

wherein R¹ and R² are each independently a peptidyl moiety; and wherein R and R² are optionally joined together to form an intramolecularly conjugated protein

### Embodiment 14

The protein of Embodiment 13, wherein the protein is of Formula (I).

### Embodiment 15

The protein of Embodiment 13, wherein the protein is of Formula (II).

### Embodiment 16

The protein of Embodiment 13, wherein the protein is of Formula (III).

### Embodiment 17

The protein of any one of Embodiments 13 to 18, wherein R¹ and R² each independently comprise a protein α-strand or a protein β-strand.

### Embodiment 18

The protein of any one of Embodiments 13 to 17, wherein t R¹ and R² are joined together to form an intramolecularly conjugated protein.

### Embodiment 19

The protein of any one of Embodiments 13 to 17, wherein R¹ and R² are not joined together.

### Embodiment 20

A pyrrolysyl-tRNA synthetase comprising at least 5 amino acid residues substitutions within the substrate-binding site of the pyrrolysyl-tRNA synthetase having the amino acid sequence of SEQ ID NO: 3.

### Embodiment 21

The pyrrolysyl-tRNA synthetase of Embodiment 20, wherein the substrate-binding site comprises residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO: 3.

### Embodiment 22

The pyrrolysyl-tRNA synthetase of Embodiment 21, wherein the at least 5 amino acid residues substitutions are a substitution for alanine at position 302, a substitution for asparagine at position 346, a substitution for cysteine at position 348, a substitution for tyrosine at position 384, and a substitution for tryptophan at position 417 as set forth in the amino acid sequence of SEQ ID NO: 3.

### Embodiment 23

The pyrrolysyl-tRNA synthetase of Embodiment 22, wherein the at least 5 amino acid residues substitutions are isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO: 3.

### Embodiment 24

The pyrrolysyl-tRNA synthetase according to any one of Embodiment 20 to 23, wherein the pyrrolysyl-tRNA synthetase has an amino acid sequence of SEQ ID NO: 1.

### Embodiment 25

The pyrrolysyl-tRNA synthetase according to any one of Embodiment 20 to 24, wherein the pyrrolysyl-tRNA synthetase is encoded by a nucleic acid sequence of SEQ ID NO: 2.

### Embodiment 26

A vector comprising a nucleic acid sequence encoding the pyrrolysyl-tRNA synthetase according to any one of Embodiments 20 to 25.

### Embodiment 27

The vector of Embodiment 26, further comprising a nucleic acid sequence encoding tRNA^{Pyl}.

### Embodiment 28

A complex comprising a pyrrolysyl-tRNA synthetase of any one of Embodiments 20 to 27 and fluorosulfate-L-tyrosine having the following formula:

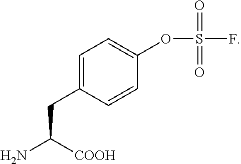 [SMILES: N[C@@H](Cc1ccc(OS(=O)(=O)F)cc1)C(=O)O]

### Embodiment 29

The complex of Embodiment 28, further comprising a tRNA^{Pyl}.

### Embodiment 30

A cell comprising the biomolecule conjugate of any one of Embodiments 1 to 12.

### Embodiment 31

A cell comprising the protein of anyone of Embodiments 13 to 19.

### Embodiment 32

A cell comprising the pyrrolysyl-tRNA synthetase of any one of Embodiments 20 to 25.

### Embodiment 33

A cell comprising the vector of Embodiment 26 or 27.

### Embodiment 34

A cell comprising the complex of Embodiment 28 or 29.

### Embodiment 35

A cell comprising fluorosulfate-L-tyrosine of the formula:

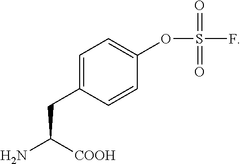 [SMILES: N[C@@H](Cc1ccc(OS(=O)(=O)F)cc1)C(=O)O]

### Embodiment 36

The cell of Embodiment 35, further comprising a pyrrolysyl-tRNA synthetase comprising at least 5 amino acid residues substitutions within the substrate-binding site of the pyrrolysyl-tRNA synthetase set forth in SEQ ID NO:3.

### Embodiment 37

The cell of Embodiment 35, further comprising a vector which comprises a nucleic acid sequence encoding a pyrrolysyl-tRNA synthetase which comprises at least 5 amino acid residues substitutions within the substrate-binding site of the pyrrolysyl-tRNA synthetase set forth in SEQ ID NO:3.

### Embodiment 38

The cell of anyone of Embodiments 35 to 37, further comprising a tRNA^{Pyl}.

### Embodiment 39

The cell of any one of Embodiments 30 to 38, wherein the cell is a bacterial cell or a mammalian cell.

### Embodiment 40

A method of forming the biomolecule conjugate of Embodiment 11, the method comprising: (i) contacting an FSY moiety within an FSY biomolecule with a compound comprising the second biomolecule moiety, wherein the second biomolecule is reactive with the FSY moiety; thereby forming the biomolecule conjugate having an intermolecular linker.

### Embodiment 41

A method of forming the biomolecule conjugate of Embodiment 12, the method comprising: (i) contacting an FSY moiety within an FSY biomolecule with a second biomolecule moiety in the FSY biomolecule, wherein the second biomolecule is reactive with the FSY moiety; thereby forming the biomolecule conjugate having an intramolecular linker.

### Embodiment 42

The method of Embodiment 40 or 41, wherein the contacting in (i) is performed within a cell.

### Embodiment 43

The method of Embodiment 40 or 41, further comprising, prior to the contacting in step (i): performing (ii) contacting a biomolecule, a pyrrolysyl-tRNA synthetase of any one of Embodiments 20 to 25, a tRNA^{Pyl}, and a fluorosulfate-L-tyrosine having the formula:

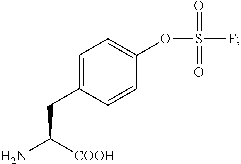 [SMILES: N[C@@H](Cc1ccc(OS(=O)(=O)F)cc1)C(=O)O]

to form the FSY biomolecule.

### Embodiment 44

The method of Embodiment 43, wherein the contacting in (ii) is performed within a cell.

### Embodiment 45

A method of forming the protein of Embodiment 18, the method comprising contacting an FSY protein with a second protein comprising lysine, histidine, or tyrosine; thereby forming the intramolecularly conjugated protein.

### Embodiment 46

A method of forming the protein of Embodiment 19, the method comprising contacting the fluorosulfate-L-tyrosine in an FSY protein with a lysine, histidine, or tyrosine in a second protein; thereby forming the intermolecularly conjugate protein.

### Embodiment 47

The method of Embodiment 45 or 46, further comprising producing the FSY protein, the method comprising contacting a protein, a pyrrolysyl-tRNA synthetase of any one of Embodiments 20 to 25, a tRNA^{Pyl}, and fluorosulfate-L-tyrosine having the formula:

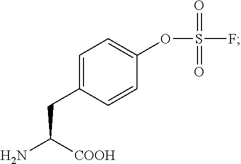 [SMILES: N[C@@H](Cc1ccc(OS(=O)(=O)F)cc1)C(=O)O]

thereby producing the FSY protein.

### Embodiment 48

The method of any one of Embodiments 40 to 47, wherein contacting comprises a sulfur-fluoride exchange reaction.

### Embodiment 49

The method of Embodiment 48, wherein contacting comprises a proximity-enabled, sulfur-fluoride exchange reaction.

### Embodiment 50

The method of any one of Embodiments 46 to 50, wherein contacting is performed within a cell.

### Embodiment 51

A protein comprising an unnatural amino acid proximal to lysine, histidine, or tyrosine; wherein the unnatural amino acid has a side chain of formula:

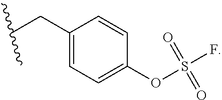 [SMILES: *Cc1ccc(OS(=O)(=O)F)cc1]

### Embodiment 52

A protein comprising a moiety of Formula (A), a moiety of Formula (B), a moiety of Formula (C), or a combination of two or more thereof:

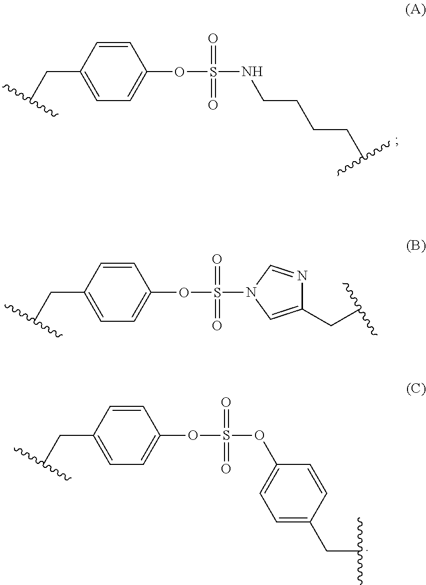 [SMILES: *CCCCNS(=O)(=O)Oc1ccc(C*)cc1 ; *Cc1ccc(OS(=O)(=O)n2cnc(C*)c2)cc1 ; *Cc1ccc(OS(=O)(=O)Oc2ccc(C*)cc2)cc1]

### Embodiment 53

A cell comprising the protein of Embodiment 51 or 52.

### Embodiments P1-P29

### Embodiment P1

A biomolecule conjugate comprising a first biomolecule moiety conjugated to a second biomolecule moiety through a bioconjugate linker, wherein said bioconjugate linker has the formula:

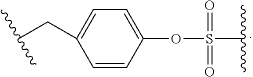 [SMILES: *Cc1ccc(OS(*)(=O)=O)cc1]

### Embodiment P2

The biomolecule conjugate of Embodiment P1, wherein said biomolecule conjugate has the formula: R¹-L¹-A-X¹-L²-R²; wherein A is said bioconjugate linker; R¹ is said first biomolecule moiety; R² is said second bioconjugate moiety; L¹ is a bond or a first covalent linker; L² is a bond or a second covalent linker; and X¹ is —NR⁵—, —O—, —S—,

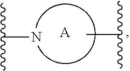

wherein ring A is a substituted or unsubstituted heteroarylene or substituted or unsubstituted heterocycloalkylene, and wherein the nitrogen in A is attached to said bioconjugate linker; and R⁵ is hydrogen, substituted or unsubstituted alkylyl, substituted or unsubstituted heteroalkylyl, substituted or unsubstituted cycloalkylyl, substituted or unsubstituted heterocycloalkylyl, substituted or unsubstituted arylyl, or substituted or unsubstituted heteroarylyl.

### Embodiment P3

The biomolecule conjugate of Embodiment P2, wherein L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{3A}, R^{3B}R^{4A}, and R^{4B} are independently hydrogen, substituted or unsubstituted alkylyl, substituted or unsubstituted heteroalkylyl, substituted or unsubstituted cycloalkylyl, substituted or unsubstituted heterocycloalkylyl, substituted or unsubstituted arylyl, or substituted or unsubstituted heteroarylyl.

### Embodiment P4

The biomolecule conjugate of Embodiment P2, wherein X is imidazole, —NH— or —O—.

### Embodiment P5

The biomolecule conjugate of Embodiment P1, wherein said first biomolecule moiety is a peptidyl moiety.

### Embodiment P6

The biomolecule conjugate of Embodiment P1, wherein said second biomolecule moiety is a peptidyl moiety.

### Embodiment P7

The biomolecule conjugate of any one of Embodiments P2 to P4, wherein -L¹-R¹ is a peptidyl moiety.

### Embodiment P8

The biomolecule conjugate of any one of Embodiments P2 to P4, wherein -L²-R² is a peptidyl moiety.

### Embodiment P9

The biomolecule conjugate of Embodiment P1, wherein said first biomolecule moiety is a nucleic acid moiety or a carbohydrate moiety.

### Embodiment P10

The biomolecule conjugate of Embodiment P1, wherein said second biomolecule moiety is a nucleic acid moiety or a carbohydrate moiety.

### Embodiment P11

The biomolecule conjugate of anyone of Embodiments P2 to P4, wherein -L¹-R¹ is a nucleic acid moiety or a carbohydrate moiety.

### Embodiment P12

The biomolecule conjugate of anyone of Embodiments P2 to P4, wherein -L²-R² is a nucleic acid moiety or a carbohydrate moiety.

### Embodiment P13

A mutant pyrrolysyl-tRNA synthetase comprising at least 5 amino acid residues substitutions within the substrate-binding site of said mutant pyrrolysyl-tRNA synthetase.

### Embodiment P14

The mutant pyrrolysyl-tRNA synthetase of Embodiment P13, wherein said substrate-binding site comprises residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO: 3.

### Embodiment P15

The mutant pyrrolysyl-tRNA synthetase of Embodiment P14, wherein said at least 5 amino acid residues substitutions are a substitution for alanine at position 302, a substitution for asparagine at position 346, a substitution for cysteine at position 348, a substitution for tyrosine at position 384, and a substitution for tryptophan at position 417 as set forth in the amino acid sequence of SEQ ID NO: 3.

### Embodiment P16

The mutant pyrrolysyl-tRNA synthetase of Embodiment P15, wherein said at least 5 amino acid residues substitutions are isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO: 3.

### Embodiment P17

The mutant pyrrolysyl-tRNA synthetase according to anyone of Embodiments P13 to P16, wherein said mutant pyrrolysyl-tRNA synthetase has an amino acid sequence of SEQ ID NO: 1.

### Embodiment P18

The mutant pyrrolysyl-tRNA synthetase according to anyone of Embodiments P13 to P17, wherein said mutant pyrrolysyl-tRNA synthetase is encoded by a nucleic acid sequence of SEQ ID NO: 2.

### Embodiment P19

A vector comprising a nucleic acid sequence encoding a mutant pyrrolysyl-tRNA synthetase according to any one of Embodiments P13 to P18.

### Embodiment P20

The vector of Embodiment P19, further comprising a nucleic acid sequence encoding tRNA^{Pyl}.

### Embodiment P21

A complex comprising a mutant pyrrolysyl-tRNA synthetase according to any one of Embodiments P13 to P18; and fluorosulfate-L-tyrosine (FSY) having the following formula:

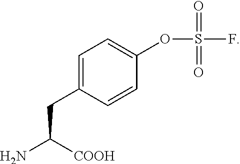 [SMILES: N[C@@H](Cc1ccc(OS(=O)(=O)F)cc1)C(=O)O]

### Embodiment P22

The complex of Embodiment P21, further comprising a tRNA^{Pyl}.

### Embodiment P23

A modified cell, comprising a biomolecule conjugate according to any one of Embodiments P1 to P12, a mutant pyrrolysyl-tRNA synthetase according to any one of Embodiments P13 to P18, a vector according to Embodiment P19 or P20, or a complex according to Embodiment P21 or P22.

### Embodiment P24

A modified cell comprising fluorosulfate-L-tyrosine (FSY) having the following formula:

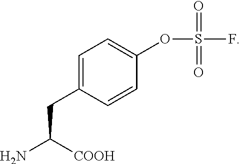 [SMILES: N[C@@H](Cc1ccc(OS(=O)(=O)F)cc1)C(=O)O]

### Embodiment P25

The modified cell of Embodiment P24, further comprising a mutant pyrrolysyl-tRNA synthetase according to any one of Embodiments P13 to P18.

### Embodiment P26

The modified cell of Embodiment P24, further comprising the vector of Embodiment P19 or P20.

### Embodiment P27

The modified cell of Embodiment P24, further comprising a tRNA^{Pyl}.

### Embodiment P28

A method of forming a biomolecule conjugate, the method comprising contacting a mutant pyrrolysyl-tRNA synthetase according to any one of Embodiments P13 to P18, a tRNA^{Pyl}, and a fluorosulfate-L-tyrosine (FSY) having the following formula:

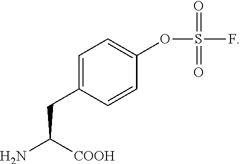 [SMILES: N[C@@H](Cc1ccc(OS(=O)(=O)F)cc1)C(=O)O]

### Embodiment P29

The method of Embodiment 28, wherein said contacting is performed within a cell.

### EXAMPLES

The following examples are intended to further illustrate certain embodiments and aspects of the disclosure. The examples are not intended to limit the spirit or scope of the disclosure or claims.

### Example 1

Genetically encoding fluorosulfate-L-tyrosine to react with lysine, histidine and tyrosine via SuFEx in proteins in vivo

Introducing new chemical reactivity into proteins in living cells would endow innovative covalent bonding ability to proteins for research and engineering in vivo. Latent bioreactive unnatural amino acids (Uaas) can be incorporated into proteins to react with target natural amino acid residues via proximity-enabled reactivity. To expand the diversity of proteins amenable to such reactivity in vivo, a chemical functionality that is biocompatible and able to react with multiple natural residues under physiological conditions is highly desirable. Here the inventors report the genetic encoding of fluorosulfate-L-tyrosine (FSY), the first latent bioreactive Uaa that undergoes sulfur-fluoride exchange (SuFEx) on proteins in vivo. FSY was found nontoxic to E. coli and mammalian cells; after being incorporated into proteins, it selectively reacted with proximal lysine, histidine, and tyrosine via SuFEx, generating covalent intra-protein bridge and inter-protein crosslink of interacting proteins directly in living cells. The proximity-activatable reactivity, multi-targeting ability, and excellent biocompatibility of FSY will be invaluable for covalent manipulation of proteins in vivo. Moreover, genetically encoded FSY hereby empowers general proteins with the next generation of click chemistry, SuFEx, which will afford broad utilities in chemical biology, drug discovery, and biotherapeutics.

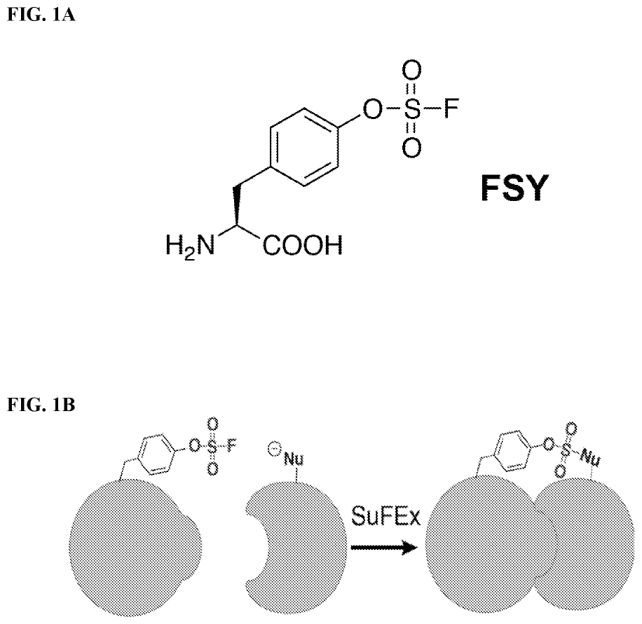

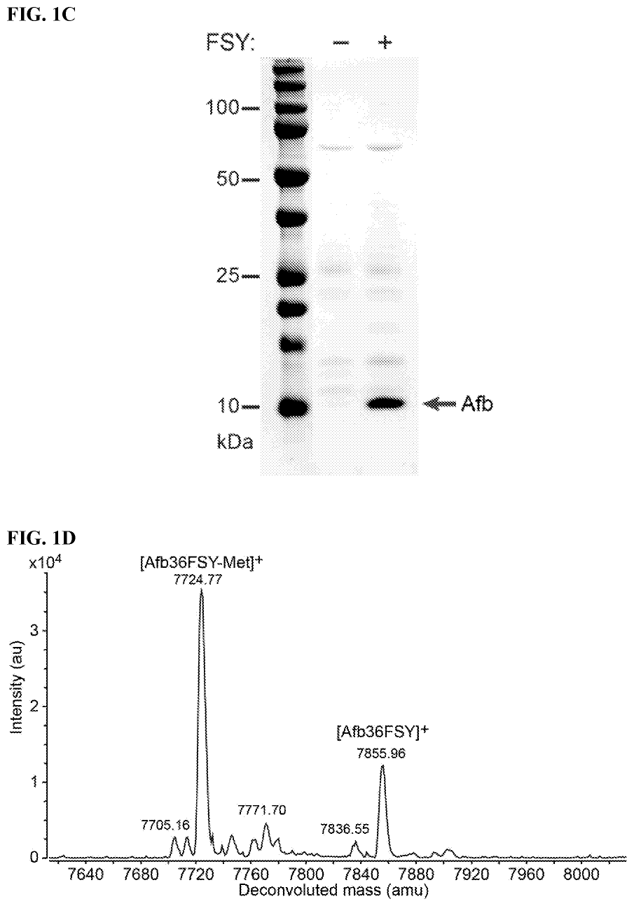

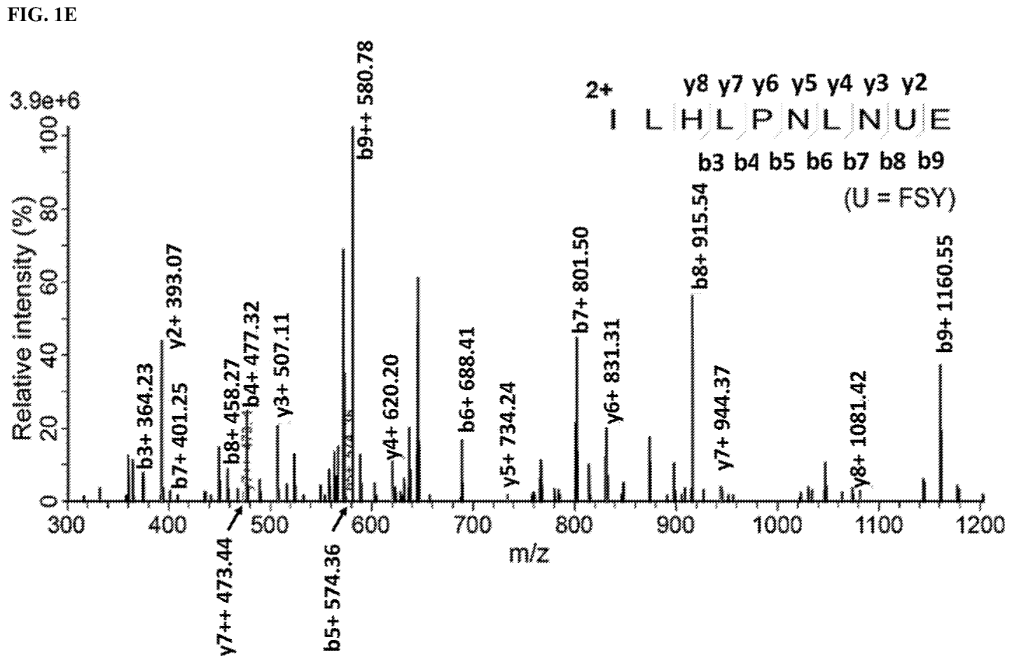

A new tRNA-synthetase pair was developed to genetically incorporate fluorosulfate-L-tyrosine (FSY)(FIG. 1A) into proteins in E. coli and mammalian cells. The inventors found that FSY was generally nontoxic to cells, and was able to react with Lys, His, and Tyr via proximity-enabled SuFEx reaction within and between proteins under physiological conditions (FIG. 1). The inventors demonstrated the crosslinking of interacting proteins using FSY directly in vivo.

FSY was synthesized using the SO₂F₂/borax method (88% yield). Dong et al, Angew. Chem. Int. Ed. Engl, 53:9430-9448 (2014); Chen et al, Angew. Chem. Int. Ed. Engl., 55:1835-1838 (2016). To genetically encode FSY, the inventors developed a mutant pyrrolysyl-tRNA synthetase (PylRS) specific for FSY. A PylRS mutant library was generated by mutating residues Ala302, Leu305, Tyr306, Leu309, Ile322, Asn346, Cys348, Tyr384, Val401, and Trp417 of the Methanosarcina mazei PylRS using the small-intelligent mutagenesis approach, and subjected to selection as described. Lacey et al, ChemBioChem, 14:2100-2105 (2013); Wang et al, Angew. Chem. Int. Ed. Engl., 44:34-66 (2005); Takimoto et al, ACS Chem. Biol., 6:733-743 (2011). Six hits showing FSY-dependent phenotype were identified; they all converged on the same amino acid sequence (302I/346T/348I/384L/417K) which is referred to herein as FSYRS.

The incorporation specificity of FSY into proteins in E. coli was evaluated. The Zₛₚₐ affibody (Afb) gene containing a TAG codon at position 36 (Afb-36TAG) was co-expressed with the tRNA^{Pyl}/FSYRS pair in E. coli. In the absence of FSY, no full-length Afb was detected; when 1 mM FSY was added in growth media, full-length Afb36FSY was produced with a yield of 1.6 mg/L (FIG. 1C). The purified Afb36FSY was analyzed by electrospray ionization time-of-flight mass spectrometry (ESI-TOF MS) (FIG. 1D). A peak observed at 7855.96 Da corresponds to intact Afb containing FSY at site 36 (Afb36FSY: expected 7856.69 Da). A peak measured at 7724.77 Da corresponds to Afb36FSY lacking the initiating Met (Afb36FSY-Met: expected 7725.50 Da). Two minor peaks observed at 7836.55 and 7705.16 Da correspond to Afb36FSY lacking F (expected 7836.69 Da) and Afb36FSY-Met lacking F (expected 7705.49 Da), respectively, suggesting slight F elimination during MS measurement. Notably, no peaks corresponding to Afb containing other amino acids at position 36 were observed. FSY was also incorporated at position 24 of the Z protein and analyzed with tandem MS. A series of b and y ions unambiguously indicated that FSY was incorporated at the TAG-specified position 24 (FIG. 1E). The presence of 1 mM FSY did not affect E. coli growth (FIG. 6), indicating no obvious cytotoxicity. These results indicated that the evolved tRNA^{Pyl}/FSYRS pair was able to incorporate FSY with high efficiency and specificity in E. coli.

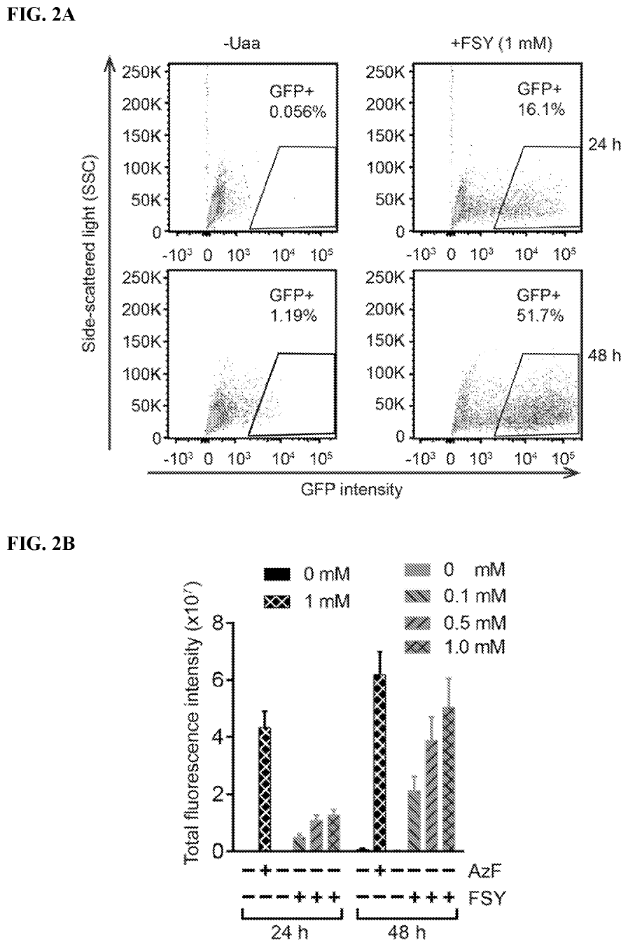

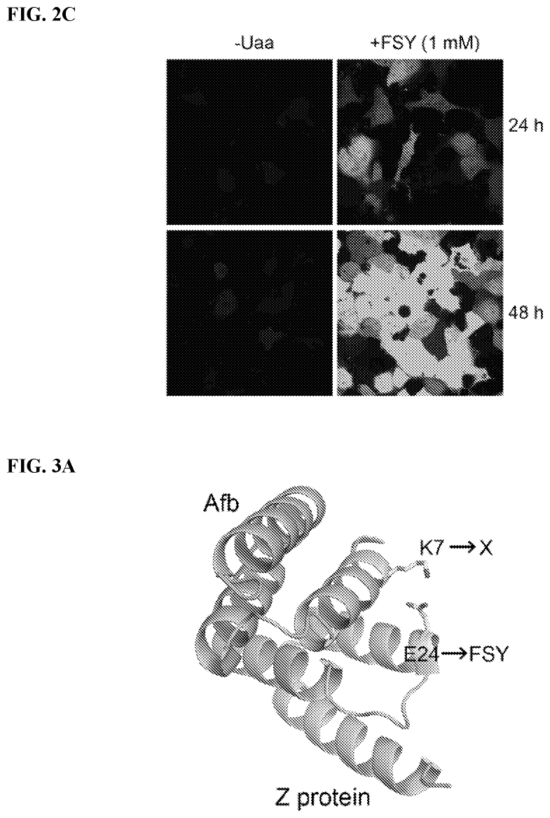

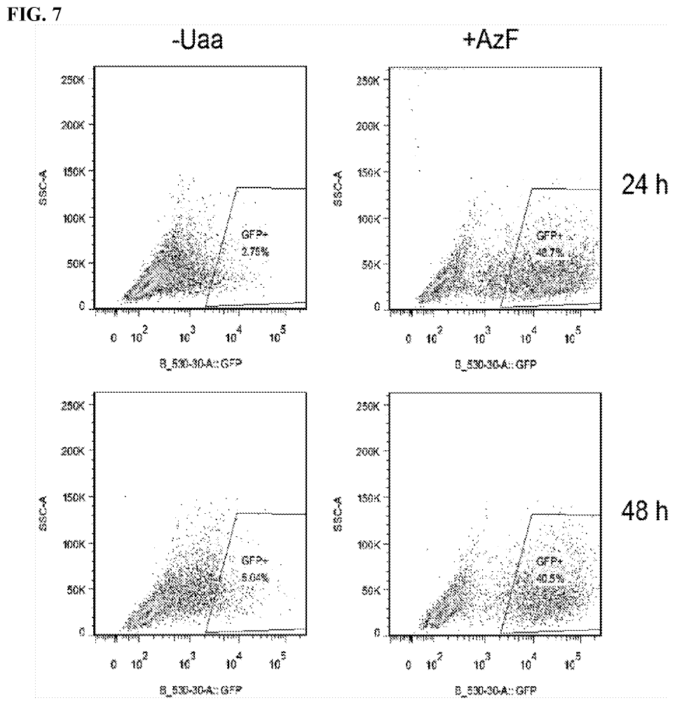

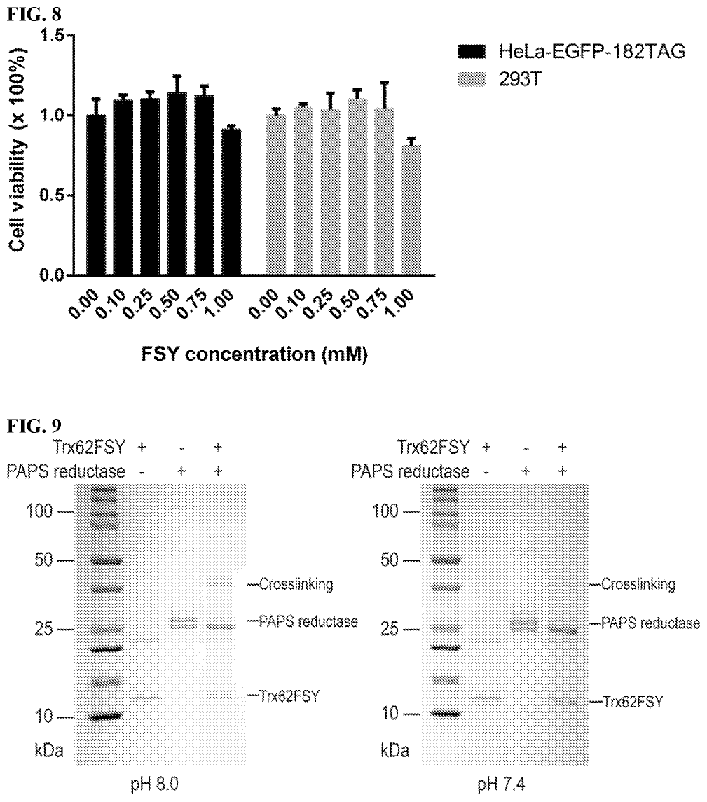

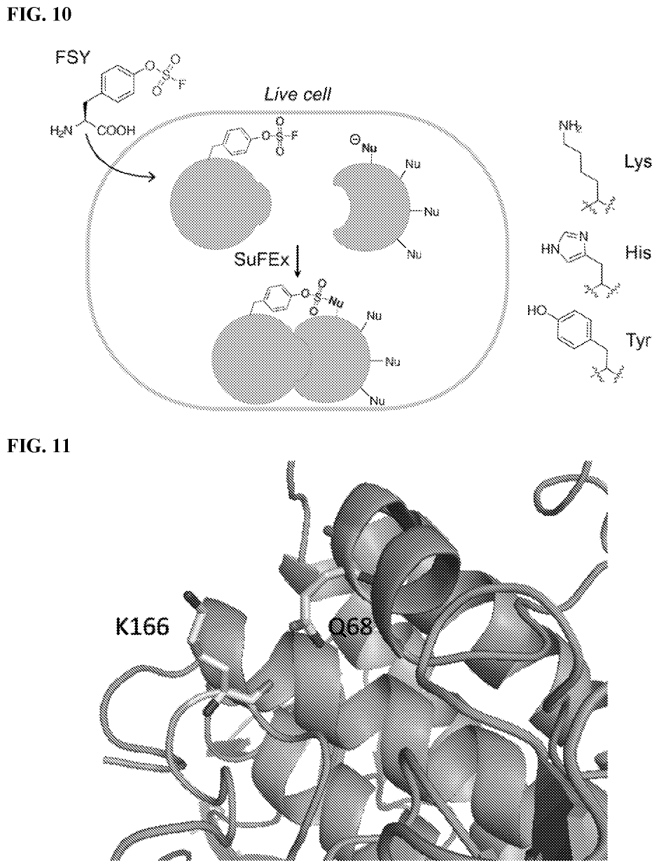

FSY incorporation into proteins in mammalian cells was tested. HeLa-EGFP-182TAG reporter cells were transfected with plasmid pMP-FSYRS-3×tRNA, which expresses FSYRS and tRNA^{Pyl} genes. Wang et al, Nat. Neurosci., 10:1063-1072 (2007). Suppression of the 182TAG codon would produce full-length EGFP rendering cells fluorescent. After transfection, cells were incubated with FSY of various concentrations at 37° C. for 24 or 48 h followed by flow cytometry. Strong EGFP fluorescence was measured from cells only when FSY was added (FIG. 2A). The fluorescence intensity increased with FSY concentration and incubation time (FIG. 2B, FIG. 7). As a positive control, p-azido-L-phenylalanine (AzF) was incorporated into reporter cells in parallel using plasmid pIre-Azi3, which is the most efficient Uaa incorporation system in mammalian cells in our hands. Coin et al, Cell, 155:1258-1269 (2013). FSY incorporation compared favorably with AzF, reaching 76% of the AzF level. Notably, while cellular toxicity is often an issue with bioreactive Uaas, no obvious toxicity of FSY to HeLa or 293T cells was observed (FIG. 8), a valuable characteristic of FSY possibly due to the extremely low background reactivity of aryl fluorosulfate inside cells. Chen et al, J. Am. Chem. Soc., 138:7353-7364 (2016). These results were also confirmed by fluorescence confocal microscopy (FIG. 2C). In the presence of FSY, strong EGFP fluorescence was observed throughout the cells, and cell morphology remained normal. No fluorescence signal was detected when FSY was not added. These results demonstrate that FSY was incorporated into proteins in mammalian cells with high efficiency and specificity without causing detrimental effects.

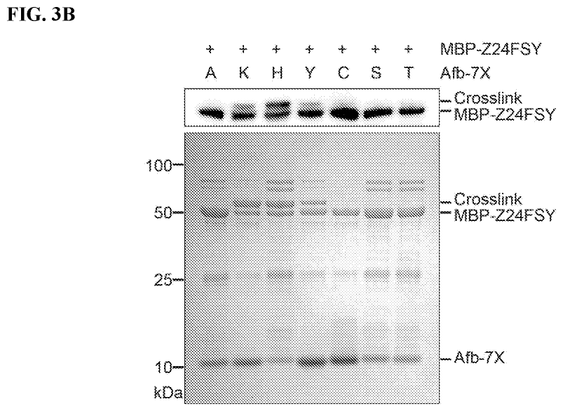

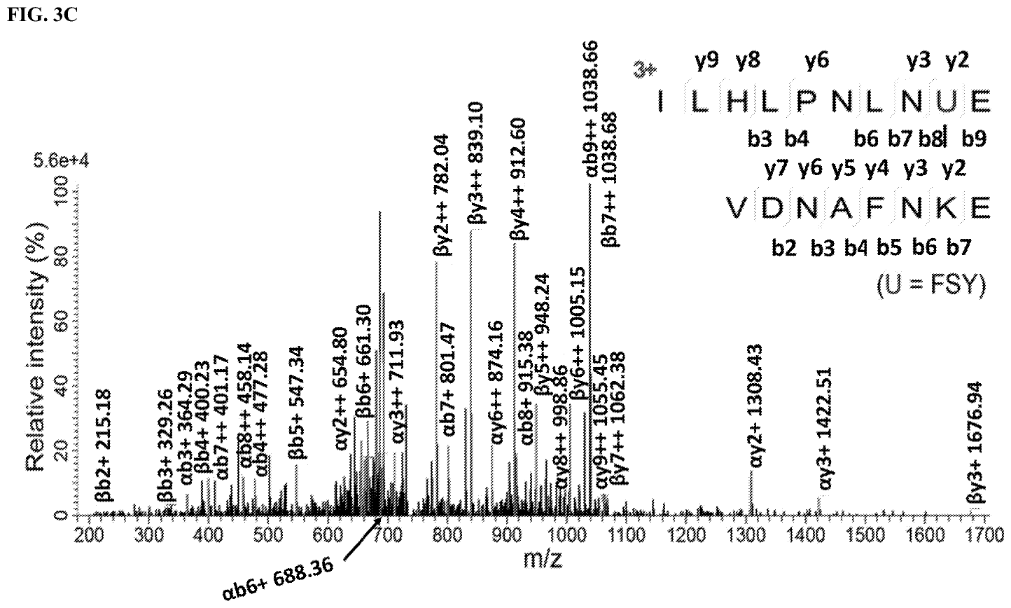

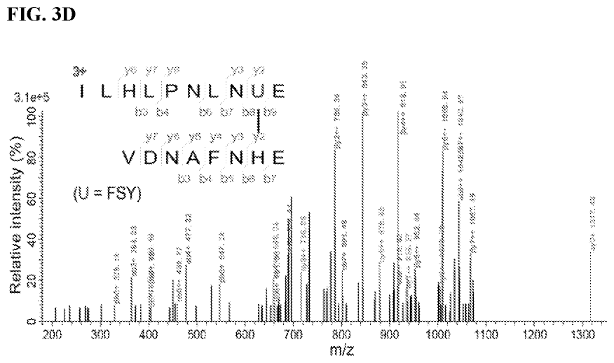

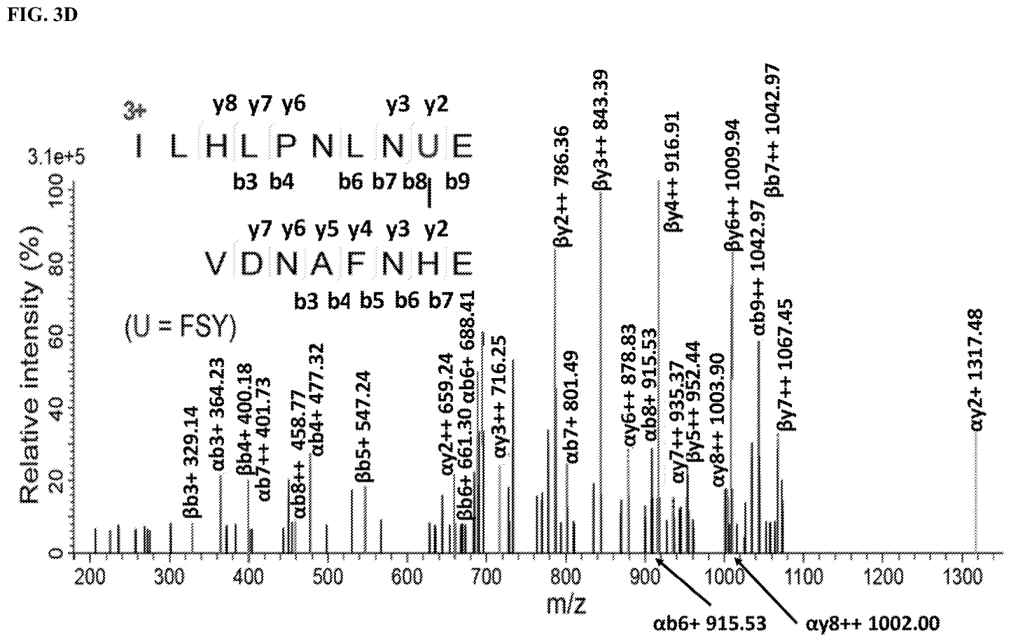

The inventors then determined whether the incorporated FSY could react with natural amino acid residues via proximity-enabled reactivity directly in E. coli. Afb binds to its substrate Z protein with a moderate affinity, providing a suitable protein framework to study FSY crosslinking in vivo. In light of the crystal structure of Afb-Z complex (Hogbom, et al, P. Proc. Natl. Acad. Sci. USA, 100:3191-3196 (2003)), the inventors introduced FSY at position 24 of Z protein and the target natural residue at position 7 of Afb, placing the two residues in close proximity upon Afb-Z binding (FIG. 3A). As aryl fluorosulfate is a weak electrophile, the inventors decided to test FSY's reactivity toward Lys, His, Tyr, Cys, Ser, and Thr using Ala as a negative control. To better separate the Afb and Z proteins of similar molecular weights, we fused maltose binding protein (MBP) to the N-terminus of Z (MBP-Z). MBP-Z and Afb were both appended with a 6×His-tag at C-terminus. To determine whether chemical crosslinking could occur in living cells, we co-expressed MBP-Z24FSY and Afb-7X (X=target residue) in E. coli. After culturing at 37° C. for 6 h, the same number of cells were analyzed using Western blot under denatured conditions. From cells expressing Afb-7Lys, Afb-7His, or Afb-7Tyr, crosslinking bands were observed with molecular weight corresponding to MBP-Z24FSY and Afb adducts (FIG. 3B). 6×His-tagged proteins were purified from cells and analyzed with SDS-PAGE. Consistently, a protein band corresponding to the crosslinked MBP-Z with Afb was clearly observed for Afb-7Lys, Afb-7His, and Afb-7Tyr (FIG. 3B), with crosslinking efficiency of 59%, 53% and 35%, respectively. In contrast, no cross-linking bands were observed when MBP-Z24FSY was co-expressed with Afb-7Cys, Afb-7Ser, Afb-7Thr, or Afb-7Ala. While aryl carbamate requires basic pH to crosslink Lys or Tyr at Afb/Z interface in vitro (Xuan et al, Angew. Chem. Int. Ed. Engl., 56:5096-5100 (2017)), FSY was able to crosslink Lys, His or Tyr directly in live E. coli cells.

To further validate the in vivo chemical crosslinking ability of FSY, the purified proteins were analyzed using tandem MS. As expected, strong signals corresponding to the covalently-linked peptides of MBP-Z24FSY and Afb-7Lys were identified (FIG. 3C). A series of b and y fragmented ions clearly indicated that the incorporated FSY crosslinked exclusively with Lys 7 of Afb. Similar MS results were also obtained for MBP-Z24FSY co-expressed with Afb-7His, confirming FSY crosslinked with the target His7 (FIG. 3D). Meanwhile, consistent with Western and SDS-PAGE results, no crosslinked peptides of MBP-Z24FSY with Afb-7Ser, Afb-7Thr, Afb-7Cys, or Afb-7Ala were detected by tandem MS. Although crosslinking of MBP-Z24FSY with Afb-7Tyr was detected using Western and SDS-PAGE (FIG. 3B), the cross-linked peptides with tandem MS could not be identified.

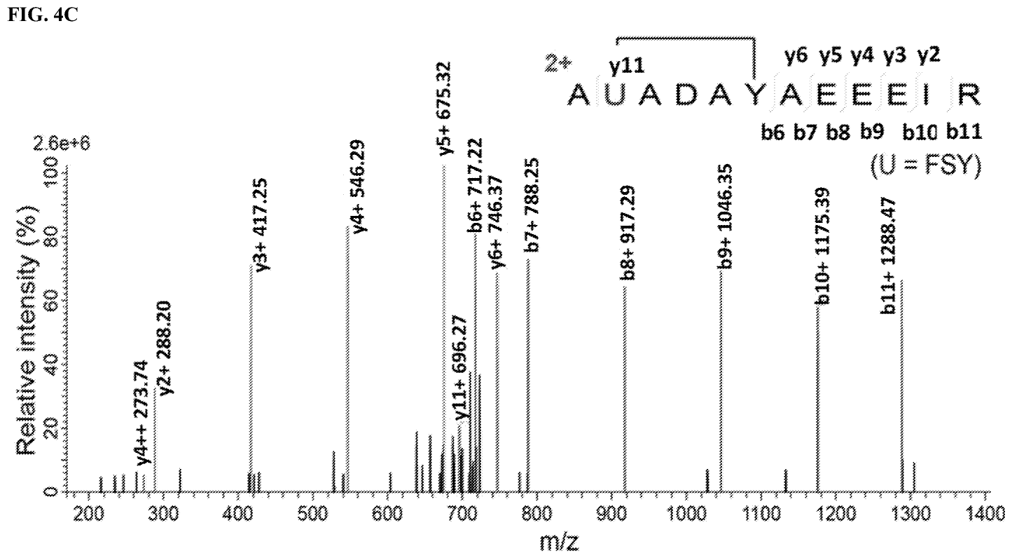

To search additional evidence of FSY reacting with Tyr, FSY and Tyr were incorporated into a single protein for intramolecular crosslinking in vivo. In E. coli the tRNA^{Pyl}/FSYRS pair was co-expressed with a mutant calmodulin gene (CaM-76TAG), which encoded 76TAG for FSY incorporation and Tyr at a nearby site 80 (FIG. 4A). This CaM protein was expressed in the presence of 1 mM FSY, purified (FIG. 4B), and analyzed with tandem MS (FIG. 4C). A series of b and y fragment ions unambiguously show that FSY formed a covalent linkage with Tyr80 via SuFEx, losing the mass of HF.

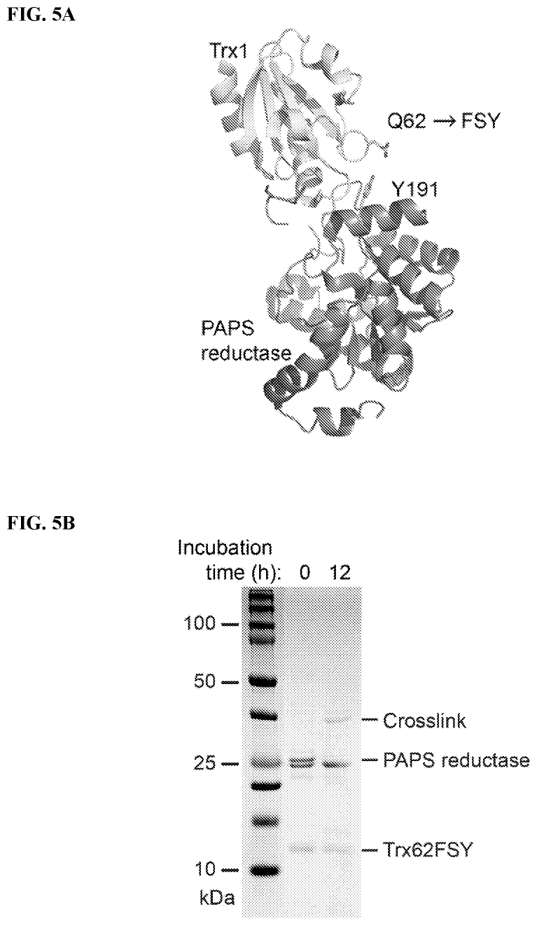

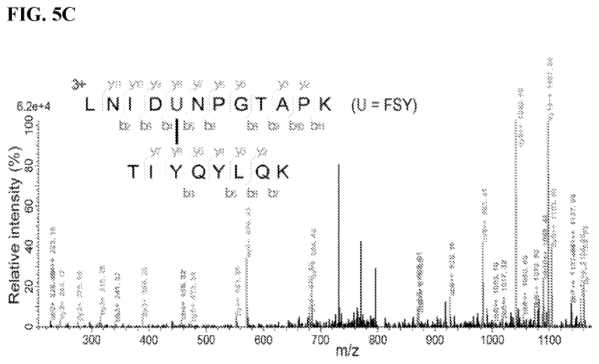

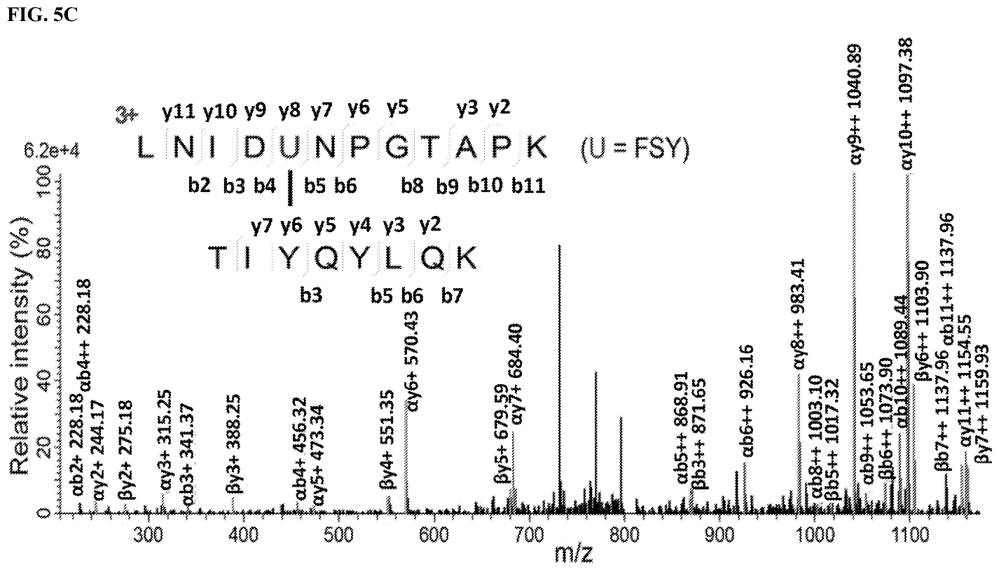

To demonstrate that FSY could crosslink interacting proteins through targeting a native Tyr residue, we tested whether FSY-armed thioredoxin (Trx) could covalently capture 3′-phosphoadenosine-5′-phosphosulfate (PAPS) reductase. Trx interacts with PAPS reductase to facilitate the reduction of adenylated sulfate to sulfite for de novo cysteine biosynthesis. Chartron et al. Biochemistry, 46:3942-3951 (2007). On the basis of the complex structure of PAPS reductase with Trx1 (Chartron et al. Biochemistry, 46:3942-3951 (2007)), FSY was incorporated into E. coli Trx1 at site 62 to target the proximal Tyr191 of PAPS reductase (FIG. 5A). Trx1(62FSY) and WT PAPS reductase were expressed, purified, and incubated in Tris buffer at pH 7.4 or 8.0 for 12 h. SDS-PAGE showed clear bands corresponding to the covalent complex of Trx1 with PAPS reductase (FIG. 5B, FIG. 9). The sample was further analyzed using tandem MS, which unambiguously indicated that FSY of Trx1 covalently crosslinked with the target Tyr191 of PAPS reductase via SuFEx reaction (FIG. 5C). Taken together, the intramolecular CaM crosslinking and intermolecular Trx1-PAPS reductase crosslinking corroborated that FSY reacted with Tyr in proximity via SuFEx reaction.

In summary, the live-cell friendly FSY was genetically encoded into proteins in E. coli and mammalian cells, which selectively reacted with proximal Lys, His and Tyr residues via SuFEx directly in live E. coli cells. Intermolecular crosslinking using bioreactive Uaas has been mainly limited to in vitro usage with few exceptions targeting Cys (Coin et al, Cell, 155:1258-1269 (2013); Yang et al, Nat. Commun., 8:2240 (2017)), FSY enables intermolecular crosslinking of interacting proteins in vivo and targeting three different residues. Since FSY's target residues are often found at protein surface and interface, FSY will dramatically expand the diversity of proteins amenable to covalent bonding and enable creative in vivo applications to exploit protein covalent bonding ability. Moreover, genetically encoding FSY now empowers proteins with the new generation of click chemistry, SuFEx, which will find broad applications in chemical biology, drug discovery, and biotherapeutics.

Materials and Methods

Chemical synthesis of FSY: The fluorosulfate-L-tyrosine HCl salt was synthesized based on the classic SO₂F₂/borax method. Chen et al, Angew. Chem. Int. Ed. Engl. 2016, 55, 1835-1838; Dong et al, Angew. Chem. Int. Ed. Engl. 2014, 53, 9430-9448.

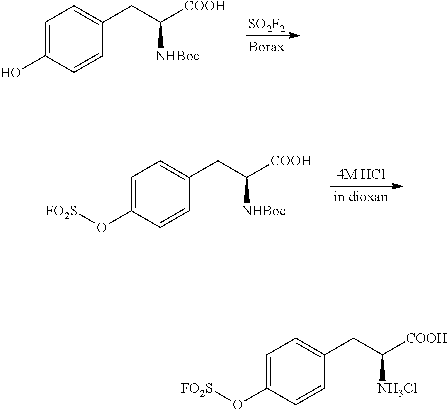

To a 2 L two-neck round-bottom flask containing a magnetic stir bar was added Boc-Tyr-OH (5.00 g, 17.8 mmol), 210 mL of CH₂Cl₂ and 860 mL of a saturated Borax solution. The mixture was stirred vigorously for 20 minutes. The reaction system was vacuumed until the biphasic solution started to degas and refilled with SO₂F₂ for three times. The reaction mixture was stirred vigorously at 25° C. overnight. CH₂Cl₂ was carefully removed using a rotary evaporator. Then 1 M aqueous HCl (210 mL) was slowly added to the reaction mixture while stirring and white solid precipitated. The mixture was filtered and the solid was washed with water (80 mL×3). The white solid was dried under vacuum (1 mm Hg) at 40° C. for 4 h affording 6.07 g (16.7 mmol) of the Boc-Tyr-OSO₂F, which was directly used in the next step without any further purification.

Boc-Tyr-OSO₂F (2.0 g, 5.5 mmol) was treated with 4 M HCl in dioxane (11 mL) and the reaction mixture was stirred overnight, during which white solid precipitated. The solid was filtered and washed by cool ether (5 mL×2), affording the targeted fluorosulfate-L-tyrosine HCl salt as a white solid (1.46 g, 88% yield). ¹H NMR (400 MHz, CD₃OD): δ (ppm) 3.23-3.41 (m, 2H), 4.32-4.34 (m, 1H), 7.45-7.53 (m, 4H); ¹³C NMR (400 MHz, CD₃OD): δ (ppm) 38.9, 57.2, 125.0, 135.3, 139.5, 153.5, 173.3; MS: 264.0 [NH₃-Tyr-OSO₂F]⁺, 286.0 [NH₂-Tyr-OSO₂F+Na]⁺

Synthetase library construction and selection: The pBK-TK3 mutant library of MmPylRS was constructed using the new small-intelligent mutagenesis approach, which uses a single codon for each amino acid and thus allows a greater number of residues to be mutated simultaneously. The following residues of MmPylRS were mutated using the procedures previously described by Lacey et al, ChemBioChem, 14:2100-2105 (2013): 302NYT, 305WTG, 306WTG/TAC, 309KYA, 322AYA, 346NDT/VMA/ATG/TGG, 348NDT/VMA/ATG/TGG, 384TTM/TAT, 401VTT, 417NDT/VMA/ATG/TGG.

DH10B cells (100 uL) harboring the pREP positive selection reporter was transformed with 100 ng of pBK-TK3 library via electroporation. The electroporated cells were immediately recovered with 1 mL of pre-warmed SOC media and agitated vigorously at 37° C. for 1 h. The recovered cells were directly plated on a LB-agar selection plate supplemented with 1 mM FSY, 12.5 g mL⁻¹ of tetracycline (Tet), 25 g mL⁻¹ of kanamycin (Kan), and 68 g mL⁻¹ of chloramphenicol (Cm). The selection plate was incubated at 37° C. for 48 h and then stored at room temperature. Colonies showing green fluorescence were diluted in 100 uL of LB and replicated on LB-agar screening plates containing 1) Tet12.5Kan25; 2) Tet12.5Kan25Cm100; 3) Tet12.5Kan25Cm100 supplemented with 1 mM FSY. After 48 h of incubation at 37° C., 6 clones present FSY-dependent fluorescence and growth were considered as hits and further characterized. The pBK plasmids encoding PylRS mutants were extracted by miniprep and then separated from reporter plasmids by DNA gel electrophoresis. The purified pBK plasmids were analyzed by Sanger-sequencing.

Plasmid Construction

pEvol-FSY: pEvol-FSY plasmid was generated by introducing the FSYRS encoding gene into pEvol vector via ligation independent cloning. Li et al, S. J. Nat. Methods, 4:251-256 (2007). Briefly, the FSYRS gene was amplified with following primers, purified, and ligated into pEvol vectors (linearized with Bgl II and Sal I) with T4 DNA polymerase. FSRYS-BglII-F is SEQ ID NO:5. FSYRS-SalI-R is SEQ ID NO:6.

pMP-3×tRNA^{Pyl}-FSYRS: The pMP-3×tRNA_{CUA}^{Pyl}-FSYRS plasmid was constructed by introducing the FSYRS gene into pMP vector via standard cloning. The FSYRS gene was amplified with following primers, digested with Nco I and Nhe I, and ligated into the pMP vector pre-treated with the same restriction enzymes. FSYRS-NcoI-F is SEQ ID NO:7. FSYRS-NheI-R is SEQ ID NO:8.

pET-Duet-Afb_{4A}-7X-MBP-Z24TAG: To evaluate the in vivo crosslinking ability of FSY, pET-Duet-Afb_{4A}-7X-MBP-Z24TAG plasmids were generated by introducing mutations at residue 7 of Afb_{4A}-7X (X=Lys, Tyr, Cys, Ser, Thr, His, or Ala) gene within the pET-Duet-MBP-Z24TAG expression vector via site-directed mutagenesis. Yang et al, Nat. Communi, 8:2240 (2017). The following primers were used. Afb-4A7A-F is SEQ ID NO:9. Afb-4A7K-F is SEQ ID NO:10.

pTak-CaM-76TAG-80Tyr: To investigate the intramolecular crosslinking ability of FSY, residue 76 and 80 of calmodulin encoding gene CaM were mutated to an amber stop codon TAG and Tyr respectively. Meanwhile, residue 75, 77, 79, 81 of CaM were mutated to Ala via overlapping PCR to assist the crosslinking reaction. The CaM gene was amplified with following primers, digested with Spe I and Blp I, and ligated into the pTak-CaM vector pre-treated with the same restriction enzymes. CaM-SpeI-F is SEQ ID NO:18. 80Tyr-R is SEQ ID NO:19. 80Tyr-F is SEQ ID NO:20.

pBad-CysH: To generate pBad-CysH plasmid, the PAPS reductase encoding gene CysH was amplified by colony PCR, digested with Nde I and Hind III, and ligated into the pBad vector pre-treated with the same restriction enzymes. CysH-NdeI-F is SEQ ID NO:22. CysH-Hind3-R is SEQ ID NO:23.

pBad-Trx35A62TAG: To generate pBad-Trx35A62TAG plasmid, residue 62 of Trx35A gene was mutated into an amber stop codon TAG using site-directed mutagenesis with following primers. Trx-62TAG-F is SEQ ID NO:24. Trx-62TAG-R is SEQ ID NO:25.

Protein Expression:

Afb36FSY: pTak-Afb36TAG-His and pBK-FSYRS were co-transformed into DH10B E. coli chemical competent cells. The transformants were plated on an LB-Kan50Cm34 agar plate and incubated overnight at 37° C. A single colony was inoculated into 5 mL of 2×YT-Kan50Cm34 and cultured overnight at 37° C. On the following day, 2 mL of overnight cell culture was diluted into 100 mL 2×YT-Kan50Cm34 and agitated vigorously at 37° C. When OD₆₀₀ reached 0.4˜0.6, half of the cell culture (50 mL) was supplemented with 1 mM FSY and 0.5 mM IPTG, then induced at 30° C. for 6 h. As a negative control, the rest 50 mL cell culture was induced with 0.5 mM IPTG at 30° C. for 6 h. Cell pellets were collected by centrifugation at 4200 g for 30 min at 4° C. and stored at −80° C.

Afb_{4A}-7X and MBP-Z24FSY: The pEvol-FSYRS and pET-Duet-Afb_{4A}-7X-MBP-Z24TAG were co-transformed into BL21(DE3) E. coli chemical competent cells. The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD₆₀₀ reached 0.4˜0.6, the cell culture was induced with 0.5 mM IPTG and 0.2% arabinose, then incubated at 37° C. for 6 h. Cell pellets were collected by centrifugation at 4200 g for 30 min at 4° C. and stored at −80° C.

CaM-76FSY-80Tyr: pBad-CaM76TAG80Tyr and pEvol-FSYRS were co-transformed into BL21(DE3) E. coli chemical competent cells. The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD₆₀₀ reached 0.4˜0.6, the cell culture was induced with 0.2% arabinose, then incubated at 37° C. for 6 h. Cell pellets were collected by centrifugation at 4200 g for 30 min at 4° C. and stored at −80° C.

Trx35A62FSY: pBad-Trx35A62TAG and pEvol-FSYRS were co-transformed into BL21(DE3) E. coli chemical competent cells. The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD₆₀₀ reached 0.4˜0.6, the cell culture was induced with 0.2% arabinose, then incubated at 30° C. for 6 h. Cell pellets were collected by centrifugation at 4200 g for 30 min at 4° C. and stored at −80° C.

PAPS reductase: pBad-CysH was transformed into DH10B E. coli chemical competent cells. The transformants were plated on an LB-Amp100 agar plate and incubated overnight at 37° C. A single colony was inoculated into 10 mL of 2×YT-Amp100 and cultured overnight at 37° C. On the following day, 10 mL of overnight cell culture was diluted into 1 L 2×YT-Amp100 and agitated vigorously at 37° C. When OD₆₀₀ reached 0.4˜0.6, the cell culture was induced with 0.2% arabinose, then incubated at 30° C. for 6 h. Cell pellets were collected by centrifugation at 4200 g for 30 min at 4° C. and stored at −80° C.

His-tag protein purification: Above cell pellets were resuspended in 14 mL lysis buffer (50 mM Tris-HCl pH 8.0, 500 mM NaCl, 20 mM imidazole, 1% v/v Tween 20, 10% v/v glycerol, lysozyme 1 mg/mL, DNase 0.1 mg/mL, and protease inhibitors). The cell suspension was lysed at 4° C. for 30 min. Cell lysate was sonicated with Sonic Dismembrator (Fisher Scientific, 30% output, 3 min, 1 sec off, 1 sec on) in an ice-water bath, followed by centrifugation (20,000 g, 30 min, 4° C.). The soluble fractions were collected and incubated with pre-equilibrated Protino®Ni-NTA Agarose resin (400 μL) at 4° C. for 1 h with constant mechanical rotation. The slurry was loaded onto a Poly-Prep® Chromatography Column, washed with 5 mL of wash buffer (50 mM Tris-HCl pH 8.0, 500 mM NaCl, 20 mM imidazole, and 10% v/v glycerol) for 3 times, and eluted with 200 μL of elution buffer (50 mM Tris-HCl, pH 8.0, 500 mM NaCl, 250 mM imidazole, and 10% v/v glycerol) for 5 times. The eluates were concentrated and buffer exchanged into 100 μL of protein storage buffer (50 mM Tris-HCl, pH 7.4 or 8.0, and 150 mM NaCl) using Amicon Ultra columns, and stored at −80° C. for future analysis.

FACS analysis of Uaa incorporation into HeLa-GFP-182TAG reporter cells: One day before transfection, 4.5×10⁴ HeLa-EGFP-182TAG reporter cells (Wang et al, Nat. Neurosci., 10:1063-1072 (2007)) were seeded in a Greiner bio-one 24 well-cell culture dish containing 500 μL of DMEM media with 10% FBS, and incubated at 37° C. in a CO₂ incubator. Plasmid pMP-3×tRNA-FSYRS (500 ng, encoding FSYRS and 3 copies of tRNA^{Pyl}) was transfected into target cells using 2.5 μL of lipofectamine 2000 following manufacturer's instructions. Six hours post transfection, the media containing transfection complex were replaced with fresh DMEM media with 10% FBS in the presence or absence of 1 mM FSY. For AzF incorporation, plasmid pIre-Azi3 (Coin et al, Cell, 155:1258-1269 (2013)) was similarly transfected and the DMEM media containing 10% FBS with or without 1 mM AzF were used. After incubation at 37° C. for 24-48 h, transfected cells were trypsinized and collected by centrifugation (1500 rpm, 5 min, r.t.). The cells were resuspended in 300 μL of FACS buffer (1×PBS, 2% FBS, 1 mM EDTA, 0.1% sodium azide, 0.28 μM DAPI) and analyzed by BD LSRFortessa™ cell analyzer.

Fluorescence confocal microscopy of HeLa-EGFP-182TAG reporter cells: One day before transfection, 4.5×10⁴ HeLa-EGFP-182TAG cells were seeded in a Greiner bio-one CELLview glass bottom dish containing 500 μL of DMEM media with 10% FBS, and incubated at 37° C. in a CO₂ incubator. Plasmid pMP-3×tRNA-FSYRS (500 ng) was transfected into target cells using 2.5 μL of lipofectamine 2000 following manufacturer's instructions. Six hours post transfection, the media were replaced with complete DMEM media with or without 1 mM FSY. The cells were incubated at 37° C. for additional 24-48 h and imaged with Nikon Eclipse Ti confocal microscope.

Mass spectrometric analysis: Intact FSY-containing Afb were analyzed by ESI-TOF MS using an Agilent 6210 mass spectrometer coupled to an Agilent 1100 HPLC system. Two micrograms of protein samples were injected by an auto-sampler and separated on an Agilent Zorbax SB-C8 column (2.1 mm ID×10 cm length) by a reverse-phase gradient of 0-80% acetonitrile for 15 min. Mass calibration was performed right before the analysis. Protein spectra were averaged and the charge states were deconvoluted using Agilent MassHunter software.

Protein digestion and tandem mass spectrometry measurement were performed as previously described by Yang et al, Nat. Communi., 8:2240 (2017). The Afb/MBP-Z samples were digested with Glu-C. The CaM and Trx1/PAPS reductase samples were digested by trypsin. Digested peptides were analyzed with an in-line EASY-spray source and nano-LC UltiMate 3000 high-performance liquid chromatography system (Thermo Fisher) interfaced with Elite mass spectrometer (Thermo Fisher). Peptides were eluted over gradient of 2%-40% buffer B (80% acetonitrile, 20% H₂O, 0.1% formic acid) at flow rate 300 nL/min from EASY-Spray PepMap C18 Columns (50 cm; particle size, 2 μm; pore size, 100 Å; Thermo Fisher). For different samples, slight modifications were made to the separation method. The Elite mass spectrometer was operated in data-dependent mode with one full MS scan at R=60,000 (m/z=200) mass range from 375 to 1800 (AGC target 1×10⁶), followed by ten CID MS/MS scans. A dynamic exclusion time of 30 s was used, and singly charged ions were excluded. Mass spectrometry raw data was searched by Maxquant.

### Example 2

FSY was used to covalently crosslink a ligand to its native receptor. Human growth hormone (hGH) is a hormone secreted by the anterior pituitary. hGH binds with the hGH receptor and stimulates growth, cell reproduction, and cell regeneration in humans. It also stimulates production of insulin-like growth factors. hGH is an interesting therapeutic target because growth hormone deficiency affects 1:4000 children in the US and it is expensive to treat (Stanley, T. Curr. Opin. Endocrinol Diabetes Obes. 2012, 19. 47-52). In addition, excess hGH has been implicated in breast cancer development, progression, and metastasis (Subramani, R. et al. Endocrinology, 6:1543-1555 (2017)).

Based on the crystal structure of hGH binding with its receptor, FSY was genetically incorporated into hGH at site 68 to target residue Lys166 of the receptor (FIG. 11). After expressing hGH(FSY68) in E. coli followed by purification, the hGH(FSY) was incubated with the extracellular domain of the hGH receptor in PBS buffer for different durations of time. The reaction mixture was then separated by SDS-PAGE, and detected using Western blot with an antibody specific for His×6 tag appended at the C-terminus of hGH.

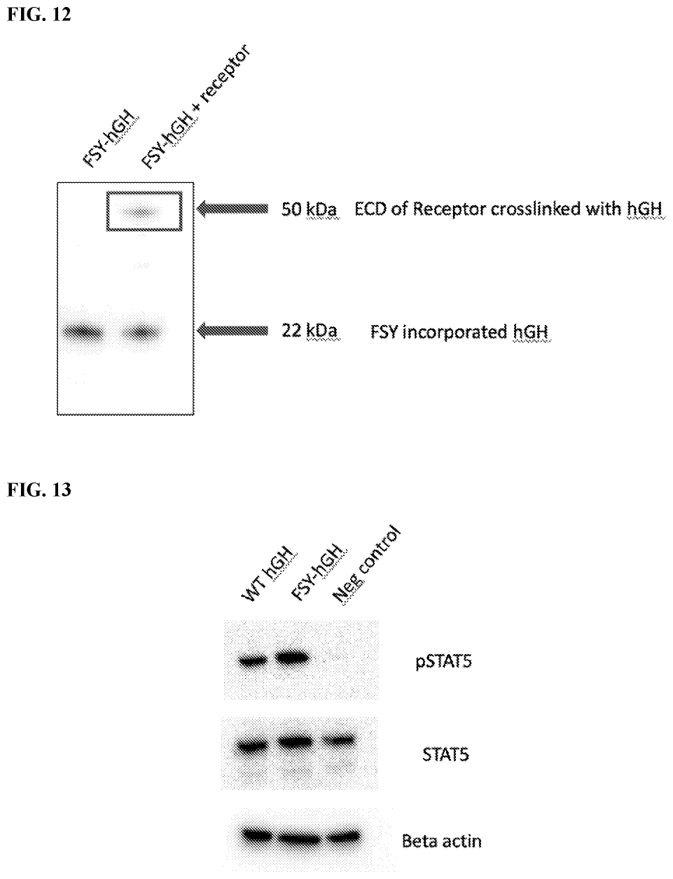

As shown in FIG. 12, hGH(FSY) was covalently crosslinked with the hGH receptor, indicated by the new band at ˜50 kD. When wild-type (WT) hGH was used under the same conditions, no crosslinking band at 50 kD was detected. These results indicate that FSY incorporated in hGH enabled hGH to irreversibly bind with its receptor.

It was also examined whether FSY incorporation into hGH would affect its biological activity. Upon hGH binding with its receptor, STAT5 is phosphorylated as a downstream signal via the JAK/STAT pathway, leading to transcription of genes important for cell immunity, proliferation, and apoptosis (Waters, M J. et al. Clin. Exp. Pharmacol. Physiol. 1999, 10760-764). The inventors stimulated BAF3 cells, a cell line with hGH receptor expression, with hGH(WT) and hGH(FSY), and then probed pSTAT5 expression using Western blot analysis of cell lysates. As shown in FIG. 13, hGH(FSY) showed the same effect of stimulating STAT5 phosphorylation as the hGH(WT), whereas the negative control using PBS buffer showed no pSTAT5 production. Therefore, these results indicate that FSY incorporation into hGH did not impact the signaling ability of hGH.

Sequence Listing
(amino acid sequence of FSYR)
SEQ ID NO: 1
MDKKPLNTLISATGLWMSRTGTIHKIKHHEVSRSKIYIEMACGDHLVVN
NSRSSRTARALRHHKYRKTCKRCRVSDEDLNKFLTKANEDQTSVKVKVV
SAPTRTKKAMPKSVARAPKPLENTEAAQAQPSGSKFSPAIPVSTQESVS
VPASVSTSISSISTGATASALVKGNTNPITSMSAPVQASAPALTKSQTD
RLEVLLNPKDEISLNSGKPFRELESELLSRRKKDLQQIYAEERENYLGK
LEREITRFFVDRGFLEIKSPILIPLEYIERMGIDNDTELSKQIFRVDKN
FCLRPMLIPNLYNYLRKLDRALPDPIKIFEIGPCYRKESDGKEHLEEFT
MLTFIQMGSGCTRENLESIITDFLNHLGIDFKIVGDSCMVLGDTLDVMH
GDLELSSAVVGPIPLDREWGIDKPKIGAGFGLERLLKVKHDFKNIKRAA
RSESYYNGISTNL*
(nucleic acid (DNA) sequence of FSYR)
SEQ ID NO: 2
ATGGATAAAAAGCCTTTGAACACTCTGATTTCTGCGACCGGTCTGTGGA
TGTCCCGCACCGGCACCATCCACAAAATCAAACACCATGAAGTTAGCCG
TTCCAAAATCTACATTGAAATGGCTTGCGGCGATCACCTGGTTGTCAAC
AACTCCCGTTCTTCTCGTACCGCTCGCGCACTGCGCCACCACAAATATC
GCAAAACCTGCAAACGTTGCCGTGTTAGCGATGAGGACCTGAACAAATT
CCTGACCAAAGCTAACGAGGATCAGACCTCCGTAAAAGTGAAGGTAGTA
AGCGCTCCGACCCGTACTAAAAAGGCTATGCCAAAAAGCGTGGCCCGTG
CCCCGAAACCTCTGGAAAACACCGAGGCGGCTCAGGCTCAACCATCCGG
TTCTAAATTTTCTCCGGCGATCCCAGTGTCCACCCAAGAATCTGTTTCC
GTACCAGCAAGCGTGTCTACCAGCATTAGCAGCATTTCTACCGGTGCTA
CCGCTTCTGCGCTGGTAAAAGGTAACACTAACCCGATTACTAGCATGTC
TGCACCGGTACAGGCAAGCGCCCCAGCTCTGACTAAATCCCAGACGGAC
CGTCTGGAGGTGCTGCTGAACCCAAAGGATGAAATCTCTCTGAACAGCG
GCAAGCCTTTCCGTGAGCTGGAAAGCGAGCTGCTGTCTCGTCGTAAAAA
GGATCTGCAACAGATCTACGCTGAGGAACGCGAGAACTATCTGGGTAAG
CTGGAGCGCGAAATTACTCGCTTCTTCGTGGATCGCGGTTTCCTGGAGA
TCAAATCTCCGATTCTGATTCCGCTGGAATACATTGAACGTATGGGCAT
CGATAATGATACCGAACTGTCTAAACAGATCTTCCGTGTGGATAAAAAC
TTCTGTCTGCGTCCGATGCTGATTCCGAACTTGTACAACTATTTACGTA
AACTGGACCGTGCCCTGCCGGACCCGATCAAAATATTCGAGATCGGTCC
TTGCTACCGTAAAGAGTCCGACGGTAAAGAGCACCTGGAAGAATTCACC
ATGCTGACATTCATTCAGATGGGTAGCGGTTGCACGCGTGAAAACCTGG
AATCCATTATCACCGACTTCCTGAATCACCTGGGTATCGATTTCAAAAT
TGTTGGTGACAGCTGTATGGTGTTAGGCGATACGCTGGATGTTATGCAC
GGCGATCTGGAGCTGTCTTCCGCAGTTGTGGGCCCAATCCCGCTGGATC
GTGAGTGGGGTATCGACAAACCTAAAATCGGTGCGGGTTTTGGTCTGGA
GCGTCTGCTGAAAGTAAAACACGACTTCAAGAACATCAAACGTGCTGCA
CGTTCCGAGTCCTATTACAATGGTATTTCTACTAACCTGTAA
(wild-type amino acid sequence of Methanosarcina
mazei PylRS)
SEQ ID NO: 3
MDKKPLNTLISATGLWMSRTGTIHKIKHHEVSRSKIYIEMACGDHLVVN
NSRSSRTARALRHHKYRKTCKRCRVSDEDLNKFLTKANEDQTSVKVKVV
SAPTRTKKAMPKSVARAPKPLENTEAAQAQPSGSKFSPAIPVSTQESVS
VPASVSTSISSISTGATASALVKGNTNPITSMSAPVQASAPALTKSQTD
RLEVLLNPKDEISLNSGKPFRELESELLSRRKKDLQQIYAEERENYLGK
LEREITRFFVDRGFLEIKSPILIPLEYIERMGIDNDTELSKQIFRVDKN
FCLRPMLAPNLYNYLRKLDRALPDPIKIFEIGPCYRKESDGKEHLEEFT
MLNFCQMGSGCTRENLESIITDFLNHLGIDFKIVGDSCMVYGDTLDVMH
GDLELSSAVVGPIPLDREWGIDKPWIGAGFGLERLLKVKHDFKNIKRAA
RSESYYNGISTNL*
(nucleic acid sequence of tRNA^{Pyl})
SEQ ID NO: 4
ggaaacctgatcatgtagatcgaatggactctaaatccgttcagccggg
ttagattcccggggtttccg
SEQ ID NO: 5 is
CTAACAGGAGGAATTAGATCTATGGATAAAAAGCCT
SEQ ID NO: 6 is
GATGATGATGATGATGGTCGACTTACAGGTTAGTAGAA
SEQ ID NO: 7 is TATGCCATGGATAAAAAGCCTTTG
SEQ ID NO: 8 is CTATGCTAGCTTACAGGTTAGTAGA
SEQ ID NO: 9 is AACGCGGAACTATCAGTCGCCGGC
SEQ ID NO: 10 is AACAAAGAACTATCAGTCGCCGGC
SEQ ID NO: 11 is AACTGCGAACTATCAGTCGCCGGC
SEQ ID NO: 12 is AACAGCGAACTATCAGTCGCCGGC
SEQ ID NO: 13 is AACACCGAACTATCAGTCGCCGGC
SEQ ID NO: 14 is AACCATGAACTATCAGTCGCCGGC
SEQ ID NO: 15 is GAACGCGTTGTCTACCATGGTATATCTCC
SEQ ID NO: 16 is
CCATGGTAGACAACGCGTTCAACTATGAACTATCAGTCGCC
SEQ ID NO: 17 is
TATATCTCCTTCTTAAAGTTAAACAAAATTATTTCTAGAGGGG
SEQ ID NO: 18 is AACTATGACTAGTCATGACCAACTGAC
SEQ ID NO: 19 is
CGCATACGCGTCCGCCTACGCTCTAGCCATCATAGT
SEQ ID NO: 20 is
TGGCTAGAGCGTAGGCGGACGCGTATGCGGAAGAGGAAATCCG
SEQ ID NO: 21 is CCAAGCTCAGCTTATTAGTGATGGTGATG
SEQ ID NO: 22 is TATACATATGTCCAAACTCGATCTAAACG
SEQ ID NO: 23
AGCCAAGCTTTTAATGATGATGATGATGATGCCCTTCGTGTAACCCACA
TTCC
SEQ ID NO: 24 is GAACATCGATTAGAACCCTGGCAC
SEQ ID NO: 25 is AGTTTTGCAACGGTCAGTTTG

What is claimed is:

1. A biomolecule conjugate comprising a first biomolecule moiety conjugated to a second biomolecule moiety through a bioconjugate linker, wherein the bioconjugate linker has the formula:

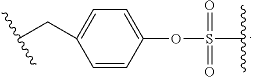 [SMILES: *Cc1ccc(OS(*)(=O)=O)cc1]

2. The biomolecule conjugate of claim 1, wherein the biomolecule conjugate has the formula:
R¹-L¹-A-X¹-L²-R²;
wherein:
A is the bioconjugate linker;
R¹ is the first biomolecule moiety;
R² is the second biomolecule moiety;
L¹ is a bond or a first covalent linker;
L² is a bond or a second covalent linker; and
X¹ is —NR⁵—, —O—, —S—, or 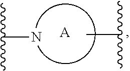
wherein ring A is a substituted or unsubstituted heteroarylene or substituted or unsubstituted heterocycloalkylene, and wherein the nitrogen in A is attached to the bioconjugate linker; and
R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R¹ and R² are optionally joined together to form an intramolecularly conjugated biomolecule conjugate.

3. The biomolecule conjugate of claim 2, wherein
L¹ is a bond, —S(O)₂—, —NR^{3A}—, —O—, —S—, —C(O)—, —C(O)NR^{3A}—, —NR^{3A}C(O)—, —NR^{3A}C(O)NR^{3B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —S(O)₂—, —NR^{4A}—, —O—, —S—, —C(O)—, —C(O)NR^{4A}—, —NR^{4A}C(O)—, —NR^{4A}C(O)NR^{4B}—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R^{3A}, R^{3B}, R^{4A}, and R^{4B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

4. The biomolecule conjugate of claim 2, wherein X¹ is —NH—, —O—, or imidazolylene.

5. The biomolecule conjugate of claim 1, wherein the first biomolecule moiety is a peptidyl moiety, a nucleic acid moiety, or a carbohydrate moiety.

6. The biomolecule conjugate of claim 5, wherein the first biomolecule moiety is a peptidyl moiety; and wherein the peptidyl moiety is covalently bonded to the bioconjugate linker via lysine, histidine, or tyrosine.

7. The biomolecule conjugate of claim 1, wherein the second biomolecule moiety is a peptidyl moiety, a nucleic acid moiety, or a carbohydrate moiety.

8. The biomolecule conjugate of claim 7, wherein the second biomolecule moiety is a peptidyl moiety; and wherein the peptidyl moiety is covalently bonded to the bioconjugate linker via lysine, histidine, or tyrosine.

9. The biomolecule conjugate of claim 2, wherein -L¹-R¹ is a peptidyl moiety, a nucleic acid moiety, or a carbohydrate moiety.

10. The biomolecule conjugate of claim 2, wherein -L²-R² is a peptidyl moiety, a nucleic acid moiety, or a carbohydrate moiety.

11. The biomolecule conjugate of claim 1, wherein the bioconjugate linker is an intermolecular linker.

12. The biomolecule conjugate of claim 1, wherein the bioconjugate linker is an intramolecular linker.

13. A protein of Formula (I), Formula (II), or Formula (III): 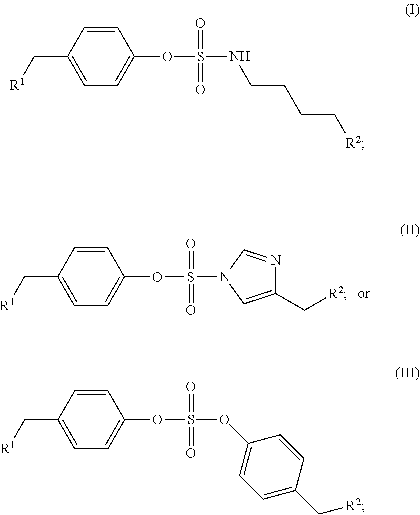
wherein R¹ and R² are each independently a peptidyl moiety; and wherein R¹ and R² are optionally joined together to form an intramolecularly conjugated protein.

14. The protein of claim 13, wherein the protein is of Formula (I).

15. The protein of claim 13, wherein the protein is of Formula (II).

16. The protein of claim 13, wherein the protein is of Formula (III).

17. The protein of claim 13, wherein R¹ and R² each independently comprise a protein α-strand or a protein β-strand.

18. The protein of claim 13, wherein t R¹ and R² are joined together to form an intramolecularly conjugated protein.

19. The protein of claim 13, wherein R¹ and R² are not joined together.

20. A pyrrolysyl-tRNA synthetase comprising at least 5 amino acid residues substitutions within the substrate-binding site of the pyrrolysyl-tRNA synthetase having the amino acid sequence of SEQ ID NO:3.

21. The pyrrolysyl-tRNA synthetase of claim 20, wherein the substrate-binding site comprises residues alanine at position 302, leucine at position 305, tyrosine at position 306, leucine at position 309, isoleucine at position 322, asparagine at position 346, cysteine at position 348, tyrosine at position 384, valine at position 401 and tryptophan at position 417 as set forth in the amino acid sequence of SEQ ID NO:3.

22. The pyrrolysyl-tRNA synthetase of claim 21, wherein the at least 5 amino acid residues substitutions are a substitution for alanine at position 302, a substitution for asparagine at position 346, a substitution for cysteine at position 348, a substitution for tyrosine at position 384, and a substitution for tryptophan at position 417 as set forth in the amino acid sequence of SEQ ID NO:3.

23. The pyrrolysyl-tRNA synthetase of claim 22, wherein the at least 5 amino acid residues substitutions are isoleucine for alanine at position 302, threonine for asparagine at position 346, isoleucine for cysteine at position 348, leucine for tyrosine at position 384, and lysine for tryptophan at position 417 as set forth in the amino acid sequence of SEQ ID NO:3.

24. The pyrrolysyl-tRNA synthetase of claim 20, wherein the pyrrolysyl-tRNA synthetase has an amino acid sequence of SEQ ID NO:1.

25. The pyrrolysyl-tRNA synthetase of claim 20, wherein the pyrrolysyl-tRNA synthetase is encoded by a nucleic acid sequence of SEQ ID NO:2.

26. A vector comprising a nucleic acid sequence encoding the pyrrolysyl-tRNA synthetase of claim 20.

27. The vector of claim 26, further comprising a nucleic acid sequence encoding tRNA^{Pyl}.

28. A complex comprising the pyrrolysyl-tRNA synthetase of claim 20 and fluorosulfate-L-tyrosine having the following formula:

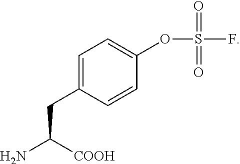 [SMILES: N[C@@H](Cc1ccc(OS(=O)(=O)F)cc1)C(=O)O]

29. The complex of claim 28, further comprising a tRNA^{Pyl}.

30. A cell comprising the biomolecule conjugate of claim 1.

31. A cell comprising the protein of claim 13.

32. A cell comprising the pyrrolysyl-tRNA synthetase of claim 20.

33. A cell comprising the vector of claim 26.

34. A cell comprising the complex of claim 28.

35. A cell comprising fluorosulfate-L-tyrosine of the formula:

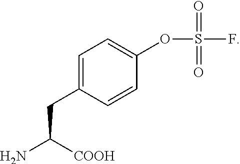 [SMILES: N[C@@H](Cc1ccc(OS(=O)(=O)F)cc1)C(=O)O]

36. The cell of claim 35, further comprising a pyrrolysyl-tRNA synthetase comprising at least 5 amino acid residues substitutions within the substrate-binding site of the pyrrolysyl-tRNA synthetase set forth in SEQ ID NO:3.

37. The cell of claim 35, further comprising a vector which comprises a nucleic acid sequence encoding a pyrrolysyl-tRNA synthetase which comprises at least 5 amino acid residues substitutions within the substrate-binding site of the pyrrolysyl-tRNA synthetase set forth in SEQ ID NO:3.

38. The cell of claim 35, further comprising a tRNA^{Pyl}.

39. The cell of claim 30, wherein the cell is a bacterial cell or a mammalian cell.

40. A method of forming the biomolecule conjugate of claim 11, the method comprising:
(i) contacting an FSY moiety within an FSY biomolecule with a compound comprising the second biomolecule moiety, wherein the second biomolecule is reactive with the FSY moiety;
thereby forming the biomolecule conjugate having an intermolecular linker.

41. A method of forming the biomolecule conjugate of claim 12, the method comprising:
(i) contacting an FSY moiety within an FSY biomolecule with a second biomolecule moiety in the FSY biomolecule, wherein the second biomolecule is reactive with the FSY moiety;
thereby forming the biomolecule conjugate having an intramolecular linker.

42. The method of claim 40, wherein the contacting in (i) is performed within a cell.

43. The method of claim 40, further comprising, prior to the contacting in step (i), performing the step:
(ii) contacting a biomolecule, a pyrrolysyl-tRNA synthetase of any one of claims 20 to 25, a tRNA^{Pyl}, and a fluorosulfate-L-tyrosine having the formula: 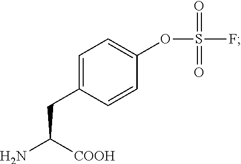 [SMILES: N[C@@H](Cc1ccc(OS(=O)(=O)F)cc1)C(=O)O]
to form the FSY biomolecule.

44. The method of claim 43, wherein the contacting in (ii) is performed within a cell.

45. A method of forming the protein of claim 18, the method comprising contacting an FSY protein with a second protein comprising lysine, histidine, or tyrosine; thereby forming the intramolecularly conjugated protein.

46. A method of forming the protein of claim 19, the method comprising contacting the fluorosulfate-L-tyrosine in an FSY protein with a lysine, histidine, or tyrosine in a second protein; thereby forming the intermolecularly conjugate protein.

47. The method of claim 45, further comprising producing the FSY protein, the method comprising contacting a protein, a pyrrolysyl-tRNA synthetase of claim 20, a tRNA^{Pyl}, and fluorosulfate-L-tyrosine having the formula: 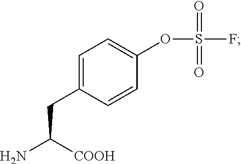 [SMILES: N[C@@H](Cc1ccc(OS(=O)(=O)F)cc1)C(=O)O]
thereby producing the FSY protein.

48. The method of claim 40, wherein contacting comprises a sulfur-fluoride exchange reaction.

49. The method of claim 48, wherein contacting comprises a proximity-enabled, sulfur-fluoride exchange reaction.

50. The method of claim 40, wherein contacting is performed within a cell.

51. A protein comprising an unnatural amino acid proximal to lysine, histidine, or tyrosine; wherein the unnatural amino acid has a side chain of formula:

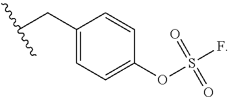 [SMILES: *Cc1ccc(OS(=O)(=O)F)cc1]

52. A protein comprising a moiety of Formula (A), a moiety of Formula (B), a moiety of Formula (C), or a combination of two or more thereof:

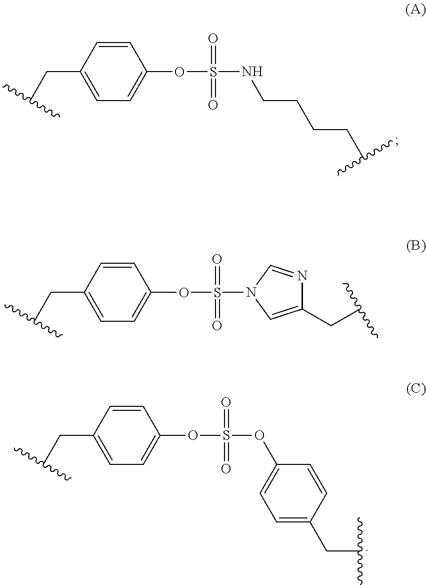 [SMILES: *CCCCNS(=O)(=O)Oc1ccc(C*)cc1 ; *Cc1ccc(OS(=O)(=O)n2cnc(C*)c2)cc1 ; *Cc1ccc(OS(=O)(=O)Oc2ccc(C*)cc2)cc1]

53. A cell comprising the protein of claim 51.
